(19) United States
(12) Patent Application Publication
(10) Pub. No.: US 2016/0082133 A1
(43) Pub. Date: Mar. 24, 2016

(54) CHEMICAL EXCHANGE SATURATION TRANSFER (CEST) BASED MRI USING REPORTER GENES AND SUBSTRATES AND METHODS THEREOF

(71) Applicant: The Johns Hopkins University, Baltimore, MD (US)

(72) Inventors: Assaf A. Gilad, Pikesville, MD (US); Amnon Bar-Shir, Baltimore, MD (US); Jeff W.M. Bulte, Fulton, MD (US); Guanshu Liu, Timomium, MD (US); Raag D. Airan, Baltimore, MD (US); Michael T. McMahon, Columbia, MD (US); Peter C. M. van Zijl, Baltimore, MD (US)

(21) Appl. No.: 14/785,495

(22) PCT Filed: Apr. 15, 2014
(86) PCT No.: PCT/US2014/034181

§ 371 Date: Oct. 19, 2015

Related U.S. Application Data
(60) Provisional application No. 61/813,933, filed on Apr. 19, 2013.

(51) Int. Cl. A61K 49/12; A61B 5/055

(52) U.S. Cl. CPC A61K 49/12; A61B 5/055

(57) ABSTRACT

The present invention features polypeptide or protein based reporters, wherein the MRI contrast is generated by the protein itself, and methods, MRI systems and MRI imaging kits related thereto. The present invention also features enzyme based reporters, wherein the contrast is generated by the substrate/product of an enzyme, and methods, MRI systems and MRI imaging kits related thereto.

### RELATED APPLICATIONS

This application claims the benefit of priority of U.S. Provisional Application No. 61/813,933, filed Apr. 19, 2013, which is hereby expressly incorporated by reference in its entirety.

### BACKGROUND OF THE INVENTION

To study proteins and enzymes in their natural context in living organisms, a noninvasive imaging technique with high spatial and temporal resolution is required. Such resolution can be achieved using magnetic resonance imaging (MRI), which has been used extensively in the last two decades for anatomical, functional, and dynamic imaging. The imaging probes for magnetic resonance imaging (MRI) are termed “contrast agents,” since they enhance the water proton-based contrast between the imaging target and the surrounding tissue. Detection with MRI relies on contrast in the MRI signal between the tissue of interest and its surrounding tissue, which can be further enhanced by expression of certain exogenous proteins that increase MRI contrast.

Recently, a new type of MRI contrast that relies on direct chemical exchange of protons with bulk water has been developed. A variety of organic molecules and lanthanide complexes possessing protons that exchange rapidly with the surrounding water protons have been suggested as powerful new contrast agents. These exchangeable protons can be “magnetically tagged” using a radiofrequency saturation pulse applied at their resonance frequency. The tagged protons exchange with the protons of surrounding water molecules and consequently reduce the MRI signal. This itself would not be visible at the low concentrations of solute, but the exchanged protons are replaced with fresh, unsaturated protons and the same saturation process is repeated. After several seconds of this process, the effect becomes amplified, and very low concentrations of agents can be detected. Hence, these agents are termed chemical exchange saturation transfer (CEST) contrast agents. Molecular MRI, with tools like chemical exchange saturation transfer (CEST) reporter genes, is attractive as it can track metabolite dynamics in vivo with better spatial resolution and anatomic co-registration than traditional techniques. In CEST MRI, a chemical group of interest is identified as one whose resonance frequency is separated sufficiently from water via a chemical shift. One main advantage of CEST MRI is the possibility of generating MRI contrast using bio-organic molecules such as polysaccharides (sugars), proteins, enzymes, and substrates that can be noninvasively detected in tissue.

CEST agents with two types of exchangeable protons that have different chemical shifts can be used to monitor pH based on ratiometric methods. In addition, a new class of CEST agents so-called DIACEST that is based only on contrast gained from diamagnetic compounds unlike agents containing a paramagnetic center and so-called PARACEST agents. In this type of agent, a lanthanide ion with a chelator binds water weakly, which can then exchange with bulk water. For example, the guanidyl and amide protons of arginine residues resonate at +1.8 and +3.6 ppm from water, respectively, with a separation from water that is sufficient for performing CEST MRI. During CEST MRI, these proton spins are saturated with radiofrequency pulses tuned to their resonance frequency. When these protons undergo chemical exchange with protons of the surrounding water, they transfer saturation to the water protons, thereby significantly reducing the signal generated by the water protons.

This new contrast mechanism has been used in a range of applications, including measuring temperature, pH, enzymatic activity, metal ions, gene expression, cell delivery, cell tracking, tumor metastasis, islet cell function, cellular activation, cell signaling, glycogen, glycosaminoglycan, glutamate, drug delivery, and kidney function.

However, there remains a need in the art for new genetically encoded reporters and substrates that can be detected by CEST MR imaging with high sensitivity, and that do not require the administration of exogenous probes or substrates, for use in CEST based MRI methods.

### SUMMARY OF THE INVENTION

As described below, the present invention features in one aspect, a method for obtaining an image by MRI, comprising introducing one or more genes into cells to be imaged, wherein the one or more genes are reporter genes or reporter substrate genes; and imaging the cells using a Chemical Exchange Saturation Transfer (CEST) based MRI technique.

In another aspect, the invention features a method for real-time monitoring of gene expression by magnetic resonance imaging (MRI), comprising introducing one or more genes into cells to be imaged, wherein the genes are reporter genes or reporter substrate genes; and imaging the cells using a CEST based MRI technique, wherein the imaging is performed in real-time.

In one embodiment, the reporter gene encodes a human protein. In a further embodiment, the human protein is human protamine-1.

In one embodiment, the reporter gene encodes an E. coli protein. In a further embodiment, the protein is a fluorescent protein. In another further embodiment, the fluorescent protein is GFP. In another related embodiment, the fluorescent protein is a superpositive GFP variant.

In one embodiment, the reporter substrate gene encodes a protein that responds to protein kinase activity. In another embodiment, the protein kinase is selected from Protein Kinase A or AKT/Protein Kinase B. In another further embodiment, the reporter substrate gene encodes a protein sequence with one or more amino acid residues selected from the group consisting of: lysine, arginine, serine and threonine. In a related embodiment, the reporter substrate gene encodes one or more tandem repeats of the sequence LRRASLG. In another further embodiment, the reporter substrate gene encodes one or more tandem repeats of the sequence RXRXX(S/T), whereas R=arginine, X=any amino acid, and Y=hydrophobic amino acid. In certain preferred embodiments, the reporter substrate gene encodes 2-12 tandem repeats, preferably 8-12 tandem repeats. In exemplary embodiments, the reporter substrate gene encodes 8 tandem repeats.

In another embodiment, the reporter gene is an endogenous gene and the reporter substrate gene is introduced into the cells to be imaged. In another embodiment, the reporter gene is introduced into the cells to be imaged, and the reporter substrate gene is an endogenous gene.

In another aspect, the invention features a method for obtaining an image by MRI, comprising introducing one or more reporter genes into cells to be imaged, wherein the reporter genes encode one or more enzymes; and imaging the cells using a CEST based MRI technique to detect substrate conversion by the enzyme.

In still another aspect, the invention features a method for real-time monitoring of gene expression by MRI, comprising introducing one or more reporter genes into cells to be imaged, wherein the reporter genes encode one or more enzymes; and imaging the cells using a CEST based MRI technique to detect substrate conversion by the enzyme, wherein the imaging is performed in real-time.

In one embodiment, the enzyme is CDase. In another embodiment, the substrate is cytosine or 5-flurocytosine.

In one embodiment, the enzyme is herpes simplex virus type-1 thymidine kinase (HSV1-TK). In another embodiment, the substrate is a nucleoside or synthetic nucleoside analog. In a further embodiment, the nucleoside analog is a synthetic thymidine analogue. In another further embodiment, the synthetic thymidine analog is 5,6-dihydrothymidine or 5-methyl-5,6-dihydrothymidine.

In one embodiment, the enzyme is Drosophila melanogaster 2′-deoxynucleoside kinase (Dm-dNK). In another embodiment, the nucleoside analog is pyrrolo-2′-deoxycytidine (pyrrolo-dC).

In one embodiment of the above-described aspects, the cells are eukaryotic cells.

In another embodiment of the above-described aspects, the reporter gene is mutated to improve MRI sensitivity.

In one embodiment, the reporter gene is an endogenous gene. In another related embodiment, expression of the endogenous gene is induced.

In one embodiment of the above-described aspects, the method is performed in vivo or in vitro.

In another aspect, the invention features a magnetic resonance imaging system comprising an imaging apparatus configured to perform a CEST MR technique; and one or more reporter genes or reporter substrate genes.

In one embodiment, the reporter gene encodes a human protein. In another embodiment, the human protein is human protamine-1.

In one embodiment, the reporter gene encodes an E. coli protein. In another embodiment, the protein is a fluorescent protein. In another related embodiment, the fluorescent protein is GFP. In another further embodiment, the fluorescent protein is a superpositive GFP variant.

In one embodiment, the reporter substrate gene encodes a protein that responds to protein kinase activity. In a further embodiment, the protein kinase is selected from Protein Kinase A or AKT/Protein Kinase B. In still another further embodiment, the reporter substrate gene encodes a protein sequence with one or more amino acid residues selected from the group consisting of: lysine, arginine, serine and threonine. In another further embodiment, the reporter substrate gene encodes one or more tandem repeats of the sequence LRRASLG.

In another aspect, the invention features a kit for MRI imaging comprising one or more reporter genes or reporter substrate genes, and instructions for use.

In still another aspect, the invention features a kit for MRI imaging comprising one or more vectors expressing one or more reporter genes or reporter substrate genes, and instructions for use.

In one embodiment of the above-described aspects, the cells are from a subject suffering from a disease or disorder. In a further embodiment, the disease or disorder is selected from the group consisting of infectious diseases, neoplasms, endocrine, nutritional, and metabolic diseases, diseases of the blood and blood-forming organs, inflammatory diseases, immune diseases, including autoimmune diseases, diseases of the nervous system, diseases of the circulatory system, diseases of the respiratory system, diseases of the digestive system, diseases of the skin, diseases of the musculoskeletal system. In a further related embodiment, the disease or disorder is selected from the group consisting of: cancer, diabetes and epilepsy.

In another aspect, the invention features a method of CEST based MRI detection of mucopolysaccharides on cells. In one embodiment, the cells are preferably cancer cells. Accordingly, in exemplary embodiment, the CEST based MRI detection of mucopolysaccharides on cells is used to assess malignancy.

In one embodiment of the above-described aspects, the MR imaging is performed in combination with positron emission tomography (PET).

### DEFINITIONS

The following terms are provided solely to aid in the understanding of this invention. These definitions should not be construed to have a scope less than would be understood by a person of ordinary skill in the art.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used in this specification and the appended claims, the singular forms “a,” “an” and “the” include plural references unless the content clearly dictates otherwise.

The term “gene” is meant to refer to a polynucleotide containing at least one open reading frame that is capable of encoding a particular polypeptide or protein after being transcribed or translated.

The term “Chemical Exchange Saturation Transfer” (CEST) is meant to refer to all saturation transfer processes between molecules that are dependent on chemical exchange between the molecules.

The term “eukaryotic cell” is meant to refer to cells from an organism whose cells contain complex structures enclosed within membranes.

The term “fluorescent protein” is meant to refer to a protein that exhibits fluorescence when exposed to light in a particular wavelength range.

The term “green fluorescent protein (GFP)” is meant to refer to a protein that exhibits bright green fluorescence when exposed to light in the blue to ultraviolet range. The term “superpositive GFP” is meant to refer to a lysine/arginine rich fluorescent protein.

The term “introduced” as used herein is meant to refer to injection, transfection or viral infection of a reporter gene or reporter substrate into a subject or cell. By “injection” is meant to refer to microinjection or introduction into the blood by, for example i.v. injection.

The term “induced” as used herein, for example as expression of a gene is induced, is meant to refer to stimulation of gene expression.

The term “magnetic resonance imaging” (MRI) is meant to refer to a noninvasive diagnostic process that uses an MR scanner to obtain images of objects, tissues, or bodies. An MR scanner uses nuclear magnetic resonance to obtain images. The MR scanner includes (1) a body-encircling magnet that generates a strong, uniform magnetic field which interacts with radio waves to excite the nuclei of specific atoms, such as hydrogen, and (2) a detector that detects relaxation of the nuclei and transforms the detected signals into a visual image.

The term “nucleic acid molecule” and “nucleotide” are used interchangeably to and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching. The term “nucleoside” is meant to refer to a nucleobase covalently attached to a sugar (ribose or deoxyribose) but without the phosphate group of a nucleotide. The term “nucleoside analog” is meant to refer to an artificial nucleoside that acts like a nucleoside in DNA synthesis.

The term “protein” or “polypeptide” is meant to refer to a polymer of amino acids. Exemplary polypeptides include gene products, naturally-occurring proteins, homologs, orthologs, paralogs, fragments, and other equivalents, variants and analogs of the foregoing.

The term “protein kinase” is meant to refer to a kinase enzyme that modifies other proteins by chemically adding phosphate groups to them (phosphorylation).

The term “real-time” is meant to refer to detection of reporter gene or reporter gene substrate 10-60 min after introduction of reporter gene or reporter gene substrate in the cell by the methods described herein.

The term “reporter” or “reporter gene” as used herein is meant to refer to any DNA sequence encoding a peptide, a protein or a polypeptide or nucleic acid that can give rise to a signal or contrast that can be detected, traced, or measured. As used in the present invention with respect to a DNA sequence, “reporter” will generally mean a cDNA sequence (although in some cases a reporter gene may have introns) that encodes a protein or polypeptide or nucleic acid that is used in the art to provide a measurable phenotype that can be distinguished over background signals. The product of said reporter gene may also be referred to a “reporter” and may be mRNA, a peptide, a polypetide, or protein, and may also be readily measured by any mRNA or protein quantification technique known in the art. A “reporter substrate gene” is meant to refer to a reporter gene that is the substrate of an enzyme. For example, the substrate of an enzyme as described herein (i.e. a kinase) acts as a reporter of enzyme activity. In certain embodiments of the invention, in addition to the gene a substrate is added in which either the substrate or the produce or both generate the contrast.

The term “subject” is meant to refer to a living multicellular vertebrate organisms, a category which includes human and veterinary subjects, for example, but not limited to, mammals; farm animals such as pigs, horses, and cows; laboratory animals such as rodents and rabbits; birds, and primates.

The term “vector” refers to a nucleic acid capable of transporting another nucleic acid to which it has been linked.

### BRIEF DESCRIPTION OF THE DRAWINGS

FIGS. 1A-1F show sequence visualization of (FIG. 1A) unphosphorylated and (FIG. 1B) phosphorylated LRRASLG peptide (phosphoryl group in orange), (FIG. 1C) CEST-spectra and (FIG. 1D) and MTRasym plots of both peptides at 1 mM. MTRasym at (FIG. 1E) 1.8 ppm and (FIG. 1F) 3.6 ppm. L 1/4 leucine; R 1/4 arginine; A 1/4 alanine; S 1/4 serine, and G 1/4 glycine. (FIG. 1C-F) Unphosphorylated (green markers) and phosphorylated (red markers).

FIG. 2 is a graph that shows biosensor phosphorylation by PKA. Shown is the time course of PKA phosphorylation measured with CEST MRI for 1 mM peptide.

FIGS. 3A-3E show design of a genetically encoded biosensor for measuring PKA activity (FIG. 3A). MTRasym of lysates of E. coli expressing the PKA biosensor gene (red) and a non-CEST-contrast-generating control (cytosine deaminase, black) (FIG. 3B). FIG. 3C shows the difference between the two spectra in b (dashed-line), with arrows pointing to peaks at 1.8 and 3.6 ppm. MTRasym maps at (FIG. 3D) 1.8 ppm and (FIG. 3E) 3.6 ppm of cell extracts of E. coli overexpressing cytosine deaminase (left capillary) and extracts of E. coli overexpressing the PKA biosensor (right capillary).

FIGS. 4A-4C show (FIG. 4A) Western blot showing hPRM1 expression. (FIG. 4B) MTRasym plots (n=3 for each cell type). (FIG. 4C) Representative MTRasym maps obtained at 1.3 ppm and 3.6 ppm frequency offsets.

FIGS. 5A and 5B show (FIG. 5A) bright-field microscopic images of the microcapsules. (FIG. 5B) T2-weighted images and overlaid MTRasym maps of microcapsules containing cells obtained at 1.3 ppm and 3.6 ppm frequency offsets.

FIGS. 6A-6F show characterization of wt-GFP and its superpositive mutants +36-GFP and +48-GFP. FIG. 6A shows fluorescence. FIG. 6B shows coomasssie-blue staining of SDS-PAGE of purified GFPs. FIG. 6C shows number of Lysine and Arginine amino acids in each GFP (total 239 amino acids). FIG. 6D shows MTRasym plots. FIG. 6E shows MTRasym maps (Δω1.5 ppm). f) MTRasym maps (Δω=3.3 ppm)

FIGS. 7A and 7B show (FIG. 7A) CDase catalyzes the deamination of cytosine and 5FC to uracil and 5FU, respectively. (FIG. 7B) A frequency-selective saturation pulse is applied to label the amine protons (cyan) of cytosine or 5FC. The labeled protons exchange with water protons, leading to a reduction in MRI signal intensity in a frequency-selective manner, generating CEST contrast.

FIGS. 8A-8D are graphs that show CEST properties of cytosine, uracil, 5FC, and 5FU at 9.4 T, pH 7.4, and 37° C. (FIGS. 8A, 8B) CEST spectra (solid lines) and MTRasym plots (dashed lines) of 40 mM (FIG. 8A) cytosine (red) and uracil (blue) and (FIG. 8B) 5FC (red) and 5FU (blue). Arrows point to the maximal MTRasym. (FIGS. 8C, 8D) Concentration dependences of MTRasym at 2 ppm and 2.4 ppm are shown for (FIG. 8C) cytosine and uracil and (FIG. 8D) 5FC and 5FU, respectively.

FIGS. 9A-9D show monitoring of recombinant CDase activity using CEST MRI. (FIG. 9A) Western blot using an anti-six-histidine-tag antibody of protein extracts from E. coli engineered to express CD or HSV1-tk (TK). (FIG. 9B) Deamination of cytosine and 5FC (20 mM) by crude CDase extracts measured using CEST MRI at 37° C. and 9.4 T. Conversion was quantified as (1−MTR^{t}_{asym}/MTR⁰_{asym})×100%, where MTR^{t}_{asym}/MTR⁰_{asym} are the contrasts at time t, and at t=0, respectively. (FIG. 9C, D) maps and statistical analysis (two tailed unpaired students t-test; n=3) of 20 mM (FIG. 9C) cytosine and (FIG. 9D) 5 FC incubated with CDase (0.6 ug crude protein) for 24 hours.

FIGS. 10A-10D show detection of CDase in mammalian cells. (FIG. 10A) Western blot of wild-type (WT) or CDase-transduced HEK293FT (293), 9L, and C17.2 cells stained with anti-CDase and anti-β-actin for total protein. (FIG. 10b) MTRasym of the supernatant of culture media of HEK293FT cells transduced with CD (CD-293) or control (WT-293) collected at different time points after incubation with 7 mM cytosine. (FIGS. 10C, D) MTRasym of the culture media 24 h after incubation with (FIG. 10C) 7 mM cytosine and (FIG. 10d) 10 mM 5FC at 2.0 and 2.4 ppm, respectively. In (FIG. 10C) and (FIG. 10D), WT and CD represent transduced and nontransduced cells respectively. * indicates p<0.05 and ** indicates p<0.01 (two-tailed unpaired Student's t test).

FIGS. 11A and 11B show imaging of cellular enzyme activity: CEST MRI of HEK293FT cells transduced with CDase (CD-293) or control cells (WT-293) en-capsulated within alginate (200-300 cells per microcapsule). (FIG. 11A) High-resolution MR image (90×50 μm, left) with corresponding microscopy (right). Scale bars=400 μm. (FIG. 11B) Conversion map overlaid on T2-weighted images, clearly showing that CD-293 cells but not WT-293 cells effectively converted 5FC to 5FU with a concurrent change in CEST signal.

FIGS. 12A-12J show evaluation of thymidine analogues as potential CEST agents. (FIG. 12A) Chemical structure of dT and compounds 1-4. (FIG. 12B) MTRasym maps of dT and 1-4 obtained at a 5 ppm frequency offset from the water resonance. (FIGS. 12C-F) CEST spectra (solid lines) and MTRasym plots (dashed lines). A B1=170 Hz was used for B-F. (FIGS. 12G-J) MTRasym plotted as a function of saturation power (B1, Hz), experimental (dots), and QUESP fitting (lines). In FIGS. 12C-J, dT is plotted in gray, 1 in red, 2 in blue, 3 in orange, and 4 in green. Data were acquired at 11.7 T, pH=7.4, and 37° C. for 20 mM CEST-agent solutions. For 1-3, MTRasym values were calculated at 5 ppm (FIGS. 12G-I), and for dT at 6 ppm (FIG. 12J).

FIGS. 13A-13D show expression, purification, and activity of HSV1-TK. (FIG. 13A) Coomassie blue staining of SDS-PAGE and western blot analysis (anti-His antibody) of purified recombinant HSV1-TK. (FIG. 13B) Relative phosphorylation of 5 sM indicated agent (dT, 1-3) in the presence of pure recombinant HSV1-TK (FIG. 13A), as measured with the Kinase-Glo assay. (FIG. 13C) CEST spectra (solid lines) and MTRasym plots (dashed lines) obtained for the kinase reactions with (black lines) and without (gray lines) purified HSV1-TK enzyme for 1. (FIG. 13D) MTRasym (5 ppm) values of 1 before (1+ATP) and after (1+ATP+HSV1-TK) phosphorylation (n=3). Relative phosphorylation is defined in the Experimental Section.

FIGS. 14A-14D show HSV1-TK specificity. Immunofluorescence of (FIG. 14A) 293^{wt} and (FIG. 14B) 293^{HSV1-tk} cells using anti-V5 antibody (red) for HSV1-TK staining overlaid on DAPI staining (blue). (FIG. 14C) Western blot of HEK-293FT cell extracts using anti-V5 antibody, with staining for HSV1-TK-expressing (HSV1-tk) and wild type (wt) cells. (FIG. 14D) Relative phosphorylation of 5 μM compounds 1 and 2 in the presence of 2 μL of cell extracts. Relative phosphorylation is defined in the Experimental Section. *p<0.05 (student's t test, unpaired, two-tailed).

FIGS. 15A-15D show imaging HSV1-TK expression. Left hemisphere: wild type 9L tumor (9L^{wt}); right hemisphere: 9L tumor expressing HSV1-TK (9L^{HSV1-tk}). (FIG. 15A) Representative longitudinal in vivo _{MTRasym} (5 ppm) maps of the mouse brain overlaid on T2-weighted images, showing the distribution of 2 obtained 1, 2, and 3 h after iv injection. (FIG. 15B) Temporal changes in the ΔMTRasym values (mean±s.d.) of each tumor type before (n=3) and after (n=8) iv administration of 2. (*p value <0.01). (FIG. 15C) Transverse views of coregistered SPECT/CT images of HSV1-TK expression, obtained 3 h after iv injection of [¹²⁵I]FIAU. (FIG. 15D) Immunostaining of perfused mouse brain section. Staining for HSV1-TK (anti-V5 antibody in red) overlaid on DAPI staining (blue) at low (4×) and high (40×) magnifications.

FIGS. 16A-16D: FIG. 16A) illustration of pyrrolo-dC phosphorylation by Dm-dNK. FIG. 16B) CEST spectra (solid lines) and MTR_{asym} plots (dashed lines) for pyrrolo-dC (red) and PBS (gray). FIG. 16C) MTR_{asym} maps of pyrrolo-dC and PBS at Dw=5.8 ppm. FIG. 16D) Fluorescence as measured from cell (9L^{wt} and 9L^{Dm-dNK}) lysate after incubation with pyrrolo-dC.

FIG. 17 (A and B) is a graph that shows liposome bearing pyrrolo-dC (Lipo-pyrrolo-dC) as compare to liposome bearing DHT (Lipo-DHT). FIG. 17A) fluorescence and FIG. 17B) CEST-MRI.

FIGS. 18A-18F show simulation of CEST spectra and MTR_{asym} plots at different magnetic field. Simulated CEST-spectra (solid lines) and MTR_{asym} plots (dashed lines) of dT (gray, FIGS. 18A-F), 1 (red, FIGS. 18A-B), 2 (blue, FIGS. 18C-D) and 3 (orange, FIGS. 18E-F). FIG. 18A (dT vs. 1), FIG. 18C (dT vs. 2) and FIG. 18E (dT vs. 3) represent the simulated CEST data for MRI scanner operating at 11.7 Tesla magnetic field. FIG. 18B (dT vs. 1), FIG. 18D (dT vs. 2) and FIG. 18F (dT vs. 3) represent the simulated CEST data for MRI scanner operating at 3 Tesla magnetic field.

FIGS. 19A-19B show CEST imaging obtained at 3.0 Tesla clinical MRI scanner. FIG. 19A) MTR_{asym} maps of PBS, dT, and 1, obtained at a 5 ppm frequency offset from the water resonance. FIG. 19B) CEST-spectra (solid lines) and MTR_{asym} plots (dashed lines) of dT (gray) and 1 (red). Arrow points to the maximal MTR_{asym} obtained from the imino proton of 1. CEST data were acquired for 20 mM CEST-agent (pH=7.4) at room temperature at 3.0 Tesla (B₁=164 Hz) MRI scanner.

FIGS. 20A and 20B show phosphorylation effect on CEST data. CEST-spectra and MTRasym plots obtained for the thymidine kinase reactions (phosphorylation) with and without purified recombinant HSV1-TK enzyme for either dT (FIG. 20A) or analog 2 (FIG. 20B) as CEST substrates. Note that the lines are overlapping, indicating no change is the CEST contrast after phosphorylation.

FIG. 21 is a graph that shows the results of a cell viability assay. The graph shows cell viability assessment of 293FT cells, transduced with HSV1-tk gene (293^{HSV1-tk}). 293^{HSV1-tk} cells were incubated with 0.5 mg/ml of either Ganciclovir (GCV) or compound 2 for 4 and 24 hours. Cell viability was determined as a percentage of treated cells compare to untreated 293^{HSV1-TK} cells (100%). Note that 2 had no toxic effect on the cells while GVC killed about 50% of the cells after 24 hour.

FIGS. 22A-22C shows Expression and activity of HSV1-TK in 9L-rat glioma cells. Immunofluorescence of FIG. 22A) 9L^{HSV1-tk} and FIG. 22B) 9L^{wt} cells using anti-V5 antibody (red) for HSV1-TK staining overlaid on a DAPI staining (blue), bar=50 μm. FIG. 22C) percentage of [¹²⁵I]FIAU uptake by control (9L^{wt}) cells and cells stably expressing the HSV1-TK (9L^{HSV1-tk}) after 1 h and 3 h incubation with 1 μCi [¹²⁵I]FIAU. *p<5×10⁻⁹ (student's t-test, unpaired, two-tailed).

FIG. 23 (A and B) are graphs that show In vivo detection of the imino proton in 9L^{HSV1-tk}. FIG. 23A) ΔMTR_{asym} plots of 9L^{wt} (blue) and 9L^{HSV1-tk} (red) (mean±s.d.; N=8 mice). The arrow pointing to the local maximal ΔMTR_{asym} represents the imino proton at 5 ppm after accumulation of 2 in 9L^{HSV1-tk} tumor. MTR_{asym} (MTR_{asym}=100×[S^{-Δω}−S^{Δω}]/S₀) was calculated from a complete CEST-spectrum (from −6 ppm to +6 ppm; S₀ image without saturation). FIG. 23B) P-values for each Δω (Student's t-test, unpaired, two-tailed) comparing the ΔMTR_{asym} of 9L^{wt} or 9L^{HSV1-tk} tumors. The lowest p-value was obtained at 5 ppm frequency offset from water (red arrow), as expected due to the accumulation of 2 solely in 9L^{HSV1-tk}. Note that second set of significant differences (p-values<0.05) between the two tumor type was found at the frequency of the hydroxyl protons (compare with MTR_{asym} plot at FIG. 18 B). In these frequencies, however, the signal is more affected by direct water saturation and the asymmetry of the conventional MT (van Zijl, P. C. et al. Magn Reson Med 2011, 65, 927).

FIGS. 24A-24C shows estimation of 2 accumulation in HSV1-tk expressing cells in vivo. FIG. 24A) Calibration curve; ΔMTR_{asym} values (at Δω=5.0 ppm) as a function of 2 concentration (mM) in PBS (11.7 Tesla, B₁=170 Hz/4000 ms, pH=7.4, and 37° C., R²=0.9997). FIG. 24B) Representative in vivo MTR_{asym} (5.0 ppm) map of the mouse brain overlaid on T₂-weighted images, showing the distribution of 2 at the last experimental time point. FIG. 24C) The calculated ΔMTR_{asym} values based on the CEST spectra MTR_{asym} (100×[S^{-5ppm}−S^{5ppm}]/S₀; mean±s.d.; N=8 mice) at 5.0 ppm for each tumor after accumulation of 2 (*p-value<0.05). The average difference between the two tumor types was approximately 0.77% (N=8, p-value=0.021). Using the calibration curve the estimated intracellular concentration of 2 is approximately 0.7 mM. The same concentration was found from simulating CEST data using the Bloch equations as described in the Methods section.

FIG. 25 is a graph that shows in vitro detection of 2 accumulation with MR spectroscopy. At least two distinctive peaks, typical to 2, appear in the spectra of the pure compound (black), a singlet at 1.22 ppm (6H of the 2 Methyls) and a triplet at 6.29 ppm (1H, the proton on 1′ carbon of the sugar). Both peaks appear in the lysate of cells incubated with the compound (red spectra) but not in the control (blue spectra). The samples were prepared using dual-phase extraction for simultaneous extraction of cellular lipids and water-soluble metabolite. Twenty five million cells (9L^{HSV1-tk}) were incubated with or without 2 for 3.5 h. After dual-phase extraction and lyophilizing of the water from the water-soluble fraction, the soluble intracellular contents were dissolved in D₂O for the ¹H-NMR experiments. Fully relaxed ¹H-NMR spectra of the extracts were acquired on a Bruker 500 MHz scanner. The signal integrals of the typical functional groups of 2 (methyl groups —(CH3)₂ at 1.22 ppm and of the CH on the C-1′ position of the sugar moiety at 6.29 ppm) and of the standard TSP (0 ppm) were determined. Then, the mM intracellular concentration of 2 was calculated as shown previously (Glunde, K. et al., Cancer Res 2005, 65, 11034) and found to be 0.37 mM.

FIG. 26 shows apoptosis assessment. Shown is monitoring apoptotic cells using TUNEL staining. Fixed brain of mouse injected with 2 (150 mg/kg body weight; 6 hours post i.v. injection) revealed very few apoptotic cells (green, overlaid on blue, DAPI staining) in both tumors (either 9L^{wt} or 9L^{HSV1-tk)}. Thus, demonstrating that 2 do not induce apoptosis in vivo.

FIG. 27 shows raw images (transverse views) of CT (left panel) and SPECT (right panel) obtained three hours after i.v. injection of [¹²⁵I]FIAU. Left hemisphere: wild type 9L tumor (9L^{wt}); right hemisphere: 9L tumor expressing HSV1-TK (9L^{HSV1-tk}).

FIG. 28 (A and B) are two graphs that show estimation of the exchange rate of cytosine (2 ppm) and 5FC (2.4 ppm) at pH=7.4 and 37° C. using the QUEST method (McMahon, M. T. et al. Magn Reson Med 2006, 55, 836). Saturation time=0.5, 1, 1.5, 2, 3, 5, and 9.5 sec, power=5.9 jtT, TR=10 sec, effective TE=41.4 msec, and RARE factor=16.

FIG. 29 depicts graphs that show the quantification of the exchange rate for cytosine using FLEX. Using the FLEX method, the exchange rate of the amine proton (at 2 ppm, red line) was found to be Kₑₓ=932 Hz.

FIGS. 30A-30C show the time course of CEST signal change in cell culture media for three cell lines in the presence of 7 mM cytosine (left panels) or 10 mM 5FC (right panels): (FIG. 30A) HEK293FT, (FIG. 30B) 9L, and (FIG. 30C) C17.2 cells. (*)p<0.05 and (**)p<0.01 (two-tailed unpaired Student's t-test).

FIGS. 31A-31D show ¹⁹F NMR spectra of (FIG. 31A) 5FC standard, (FIG. 31B) 5FU standard, and (FIG. 31C) cell culture media of HEK293 FT cells incubated with 10 mM 5FC for 24 hours. (FIG. 31D) cell culture media of 9L cells incubated with 10 mM 5FC for 48 hours.

FIG. 32 is a graph that shows percentage of viable cells 40 hours after treatment with 10 mM 5FC. WT and CD represent transduced and non-transduced cells respectively. (*)p<0.05 and (**)p<0.01 (two-tailed unpaired Student's t-test).

FIGS. 33A and 33B shows cellular CEST MRI. (FIG. 33A) MTRasym at 2.4 ppm of capsules incubated with 5FC. The CDase expressing cells (CD-293) showed conversion of 5FC, both in microcapsules and in supernatants, but not in control (WT-293) cells. (FIG. 33B) MTRasym plots of mean ROI derived from capsules or supernatants from the images in (FIG. 33A).

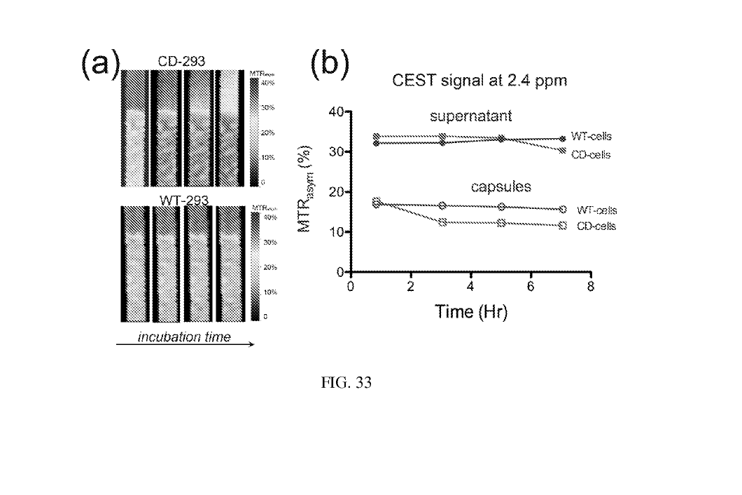

FIG. 34 is a schematic outline of mucins with different glycosylation levels.

FIG. 35A-35D show deglycosylated and untreated mucin. (FIG. 35A) shows Zspectra. (FIG. 35B) shows MTR_{asym} (FIG. 35C) shows MTR_{asym} map and (FIG. 35D) shows SDS-PAGE.

FIGS. 36A-36F show microscopy, MTw image, CEST spectra and images, and immunostaining of encapsulated lines with different MUC-1 glycosylation levels.

### DETAILED DESCRIPTION OF THE INVENTION

The present invention features polypeptide or protein based reporters, wherein the MRI contrast is generated by the protein itself, and methods, MRI systems and MRI imaging kits related thereto. The present invention also features enzyme based reporters, wherein the contrast is generated by the substrate/product of an enzyme, and methods, MRI systems and MRI imaging kits related thereto.

In one aspect, the invention features a method for obtaining an image by MRI, comprising introducing one or more genes into cells to be imaged, wherein the one or more genes are reporter genes or reporter substrate genes, and imaging the cells using a Chemical Exchange Saturation Transfer (CEST) based MRI technique.

The invention also features methods for real-time monitoring of gene expression by magnetic resonance imaging (MRI), comprising introducing one or more genes into cells to be imaged, wherein the one or more genes are reporter genes or reporter substrate genes, and imaging the cells using a CEST based MRI technique, wherein the imaging is performed in real-time.

In one embodiment, the reporter gene can be a gene that encodes a human protein. The present invention is not to be limited to any particular protein or proteins described herein. For example, in certain embodiments, the protein can be a glycoprotein It has previously been demonstrated that the arginine-rich protein human protamine-1 (hPRM1) gene can be used as a reporter gene in E. coli since it provides a higher CEST contrast than non-expressing cells (Bar-Shir, A., et al. Genetic Engineering of Human Protamine-1 for use as MRI Reporter Gene Based on Proton Exchange. in Proceedings 19th Scientific Meeting, International Society for Magnetic Resonance in Medicine 1725 (Montreal, Canada, 2011)). Accordingly, in certain embodiments, an exemplary human protein is human protamine-1.

In another embodiment, the reporter gene can encode an E. coli protein, for example a GFP or a superpositive GFP variant. GFP proteins exhibit bright green fluorescence when exposed to light in the blue to ultraviolet range.

Due to the potential for widespread usage and the evolving needs of researchers, many different mutants of GFP have been engineered. The first major improvement was a single point mutation (S65T) reported in 1995 in Nature by Roger Tsien (Heim R, et al. Nature 373 (6516): 663-4). This mutation improved the spectral characteristics of GFP, resulting in increased fluorescence, photostability, and a shift of the major excitation peak to 488 nm, with the peak emission kept at 509 nm. A 37° C. folding efficiency (F64L) point mutant to this scaffold yielding enhanced GFP (EGFP) was discovered in 1995 (Cormack B P, et al., Gene 173 (1): 33-38). EGFP allowed the practical use of GFPs in mammalian cells. Many other mutations have been made, including color mutants; in particular, blue fluorescent protein (EBFP, EBFP2, Azurite, mKalama1), cyan fluorescent protein (ECFP, Cerulean, CyPet), and yellow fluorescent protein derivatives (YFP, Citrin, Venus, YPet). BFP derivatives (except mKalama1) contain the Y66H substitution. The critical mutation in cyan derivatives is the Y66W substitution, which causes the chromophore to form with an indole rather than phenol component. Redox sensitive versions of GFP (roGFP) were engineered by introduction of cysteines into the beta barrel structure. The redox state of the cysteines determines the fluorescent properties of roGFP.

Positively charged amino acids (mostly lysine and arginine) in peptide and proteins make them attractive CEST-based contrast agents and reporter genes. Supercharged proteins, including supercharged GFP, are variants with high theoretical negative or positive charge that are resistant to aggregation. Superpositive variants (+36 and +48) were achieved by modifying the solvent-exposed amino acids to lysine or arginine (M. S. Lawrence, et al., J Am Chem Soc 129, 10110 (2007).

In certain embodiments, the MR imaging is carried out in combination with other imaging techniques, for example, but not limited to PET. In exemplary embodiments, superpositively-charged GFP, a lysine/arginine rich fluorescent protein, could allow real time monitoring of gene expression both with CEST-MRI and optical imaging, thus make it a bi-modal reporter gene.

In one embodiment, the reporter substrate gene can be a gene that encodes a protein that responds to protein kinase activity. A “reporter substrate gene” is meant to refer to a reporter gene that is the substrate of an enzyme. It is a novel finding of the present invention, that CEST imaging can be used to detect substrate conversion by the enzyme.

Protein kinases play an instrumental role in almost every signaling process in the living cell, and yet, there is no means to visualize these enzymes in vivo with high spatial and temporal resolution. Kinases are drug development targets of great importance for a wide range of diseases including cancer, diabetes, and inflammation, as each disease is linked to the perturbation of protein kinase-mediated cell signaling pathways (Noble M E et al., Protein kinase inhibitors: insights into drug design from structure. Science 2004; 303: 1800-1805). There are more than 500 different protein kinases identifies in the human genome (Manning G. et al., The protein kinase complement of the human genome. Science 2002; 298: 1912-1934).

In certain embodiments, the protein kinase is Protein kinase A (PKA). In other embodiments, the protein kinase is AKT/Protein Kinase B.

PKA, also known as cAMP-dependent protein kinase, was the first one discovered in this large family of proteins (Walsh D A et al., J Biol Chem 1968; 243:3763-3765). Since then, PKA has been extensively studied in innumerable systems, mostly in tissue extracts using antibodies or pharmacological inhibitors, which are not always specific (Murray A J et al., Pharmacological PKA inhibition: all may not be what it seems. Sci Signal 2008; 1:re4). Protein kinases catalyze the energetically irreversible step of adding a phosphoryl group (—PO4-2) to hydroxyl (—OH) targets. In particular, PKA phosphorylates the hydroxyl group of serines or threonines having the pattern RRX(S/T)Y with the highest specificity towards the peptide sequence LRRASLG (Kemp B E et al., Role of multiple basic residues in determining the substrate specificity of cyclic AMP-dependent protein kinase. J Biol Chem 1977; 252:4888-4894). This sequence has allowed the development of a genetically encoded sensor for PKA based on fluorescence resonance energy transfer (Zhang J. et al., Genetically encoded reporters of protein kinase A activity reveal impact of substrate tethering. Proc Natl Acad Sci USA 2001; 98:14997-15002), which exhibits extraordinary spatial and temporal resolution for cells in culture when using optical microscopy (Ni Q. et al., Signaling diversity of PKA achieved via a Ca2 [symbol] -cAMP-PKA oscillatory circuit. Nat Chem Biol 2011; 7:34-40). However, due to limited visible light penetration and scattering in tissue, fluorescence resonance energy transfer cannot be easily applied in vivo.

PKA is not the only protein kinase that phosphorylates consensus sequences rich in arginine, lysine, and serine residues. Among such kinases of biological importance are protein kinase C (Violin J D et al., A genetically encoded fluorescent reporter reveals oscillatory phosphorylation by protein kinase C. J Cell Biol 2003; 161:899-909) and Ca2 [symbol] /cAMP-dependent protein kinase IIa (CaMKIIa) (Kondo N et al., MALDI-TOF mass-spectrometry-based versatile method for the characterization of protein kinases. Chemistry 2009; 15:1413-1421). Even though many kinases phosphorylate sequences with high similarity in amino acid composition, the specific ordering of these amino acids determines the kinase specificity. Thus, for instance protein kinase C and CaMKII will not phosphorylate the substrate for PKA (Zhang J et al., 2001) and vice versa (Violon J D et al., 2003). This opens the possibility to engineer an array of CEST MRI-based sensors for detecting multiple variants of protein kinases in vivo.

In certain embodiments, the reporter gene can encode a protein sequence with one or more amino acid residues selected from the group consisting of: lysine, arginine, serine and threonine.

In certain embodiments, the reporter gene encodes one or more tandem repeats of the sequence RXRXX(S/T), whereas R=arginine, X=any amino acid, and Y=hydrophobic amino acid. In other embodiments, the reporter gene encodes one or more tandem repeats of the sequence LRRASLG.

CEST MRI has been used in a range of applications. Since LRRASLG contains two arginine residues coupled with a peptide (amide) bond, this peptide could serve as a good CEST agent. Moreover, a polypeptide containing multiple repeats of this sequence should yield amplification of the contrast with an overall detection sensitivity appropriate for in vivo use and a size that would yield ease of handling with standard biochemical techniques. Given that CEST contrast is highly dependent on the proton exchange rate, the presence of a strong negatively charged group, (i.e., PO₄⁻²) should slow the exchange rate and consequently reduce the contrast, making this candidate sensor able to detect phosphorylation by PKA. Accordingly, the present invention features a genetically encoded biosensor, or reporter gene, whose proton exchange rates should change sufficiently with phosphorylation by PKA to be detected with CEST MRI. Eight tandem repeats of the base peptide LRRASLG were chosen for the design of the sensor based on the above considerations. Specifically, the gene is translated to a 6.8 kDa protein that allows both high CEST contrast as well as better protein detection and purification with standard biochemical techniques. Such an MRI biosensor may have applications in monitoring stem cell and organ transplants, and screening therapeutics that target this important signaling pathway.

According to exemplary embodiments of the invention, the reporter gene is an endogenous gene and the reporter substrate gene is introduced into the cell to be images. In another exemplary embodiment, the reporter gene is introduced into the cell to be imaged, and the reporter substrate gene is an endogenous gene. The term “introduced” is meant to refer to injection or transfection of a reporter gene or reporter substrate into a subject or cell. By “injection” is meant to refer to microinjection or introduction into the blood by, for example i.v. injection. Transfection is the process by which nucleic acids are introduced into mammalian cells. Protocols and techniques are well-known in the art and include lipid transfection, electroporation, transient transfection, in vivo transfection, cotransfection, stable transfection and calcium phosphate transfection.

In one aspect, the invention features a method for obtaining an image by MRI, comprising introducing one or more reporter genes into cells to be imaged, wherein the reporter genes encode one or more enzymes; and imaging the cells using a CEST based MRI technique to detect substrate conversion by the enzyme.

In another aspect, the invention features a method for real-time monitoring of gene expression by MRI, comprising introducing one or more reporter genes into cells to be imaged, wherein the reporter genes encode one or more enzymes; and imaging the cells using a CEST based MRI technique to detect substrate conversion by the enzyme, wherein the imaging is performed in real-time.

In one embodiment, the enzyme is cytosine deaminase (CDase). CDase can be used in the methods described herein as a specific tool for noninvasive real-time imaging of enzyme activity using metal-free bio-organic diamagnetic substrates (DIACEST). CDase is expressed exclusively in bacteria and fungi as an important part of the pyrimidine salvage pathway. CDase catalyzes the conversion of cytosine to uracil through the removal of an amine group, or “deamination”. It can also convert the prodrug 5-fluorocytosine (5FC) into the chemotherapeutic agent 5-fluoruracil (5FU), making it a promising enzyme/prodrug system for cancer therapy. Because CDase activity is absent in mammalian cells, the administration of 5FC is not likely to result in significant toxicity to normal tissue.

One advantage of using CEST MRI is that there is no need to use a toxic prodrug (5FC). Instead, the CDase's natural substrate, cytosine, can be used for imaging. This allows repetitive measurements and the use of CDase as a reporter gene. Another advantage is that CEST MRI can be used to monitor multiple enzymes simultaneously as long as their substrates have exchangeable protons that resonate at distinct frequencies. This property is relevant for studying gene networks, for example, in signal transduction cascade pathways.

In another embodiment, the enzyme is herpes simplex virus type-1 thymidine kinase (HSV1-TK). Herpes simplex virus type-1 thymidine kinase (HSV1-TK) is a viral enzyme that catalyzes the synthesis of thymidine monophosphate. Compared to mammalian thymidine kinases, HSV1-TK has a lower substrate specificity and thus phosphorylates an array of therapeutics and imaging agents. These applications rely on the entrapment and accumulation of the phosphorylated nucleoside only in cells expressing HSV1-TK.1 Radiolabeled nucleosides are widely used as imaging probes for HSV1-TK expression with positron emission tomography (PET) and single photon emission computed tomography (SPECT) (Tjuvajev, J. G et al. Cancer Res. 1995, 55, 6126.; Gambhir, S. S et al. Proc. Natl. Acad. Sci. U.S.A. 1999, 96, 2333; Jacobs, A et al. Lancet 2001, 358, 727; Yaghoubi, S. S et al. Nat. Clin. Pract. Oncol. 2009, 6, 53).

The imaging probes for magnetic resonance imaging (MRI) are termed “contrast agents”, since they enhance the water proton-based contrast between the imaging target and the surrounding tissue.

CEST contrast is highly dependent on the exchange rate (kₑₓ) of the saturated protons with the water protons. In order to achieve the highest contrast and specificity, the kₑₓ should preferably be fast but in the “slow exchange regime” on the NMR time scale, as defined by kₑₓ4 Δω, where Δω is the chemical shift difference between the resonance frequency of the exchangeable protons and the water protons. (Sherry, A. D et al. 2008; Van Zijl, P. C. et al., 2011; Ward, K. M. et al., J. Magn. Reson. 2000, 143, 79). One drawback of DIACEST agents therefore is the small Δω between their exchangeable protons (i.e., Δω<4 ppm for amide, amine, guanidine, and hydroxyl protons) and the water protons. This may lead to deleterious effects from direct saturation of the water protons and increased background signal from endogenous exchangeable protons (Cai, K. et al. Nat. Med. 2012, 18, 302; Ling, W. et al.: Proc. Natl. Acad. Sci. U.S.A. 2008, 105, 2266; van Zijl, P. C et al., Proc. Natl. Acad. Sci. U.S.A. 2007, 104, 4359; Zhou, J. et al., Nat. Med. 2003, 9, 1085). The imino proton of pyrimidine-based nucleosides has a Δω=5-6 ppm, 14 making pyrimidine analogues potentially suitable for CEST imaging. However, the kₑₓ of the imino protons with water is fast (>3000 s−1) and thus needs to be reduced in order to make these nucleosides ideal CEST-based contrast agents. Changing the acid dissociation constant (pKa) value of the imino proton of thymidine (dT) by rational chemical modification can be used to modulate its kₑₓ.

In another embodiment, the enzyme is Drosophila melanogaster 2′-deoxynucleoside kinase (Dm-dNK). The Drosophila melanogaster 2′-deoxynucleoside kinase (Dm-dNK) enzyme phosphorylates a wide range of nucleoside analogs.

The substrate can be any substrate (endogenous or introduced) of the enzymes described herein. In one embodiment, the substrate is a nucleoside or synthetic nucleoside analogue. For example, the substrate can be a synthetic thymidine analogue (dT). In exemplary embodiments, dT is modified chemically to improve its CEST-MRI properties by increasing the imino proton pKa value and reducing its kₑₓ with water to comply with the slow exchange condition on the NMR time scale. In further embodiments, the synthetic dT analogue is 5-methyl-5,6-dihydrothymidine. In further embodiments, the synthetic dT analogue can be used in in vivo monitoring of HSV1-TK expression in tumors with MRI.

In other exemplary embodiments, the substrate is the fluorescent nucleoside Pyrrolo-2′-deoxycytidine (pyrrolo-dC). Preferably, pyrrolo-dC can be used for monitoring the reporter gene Dm-dNK expression with CEST MRI.

In any one of the methods described herein the cells are eukaryotic cells. In further embodiments, the cells are taken from a subject suffering from a disease or disorder. Examples of diseases or disorders include, but are not limited to infectious diseases, neoplasms, endocrine, nutritional, and metabolic diseases, diseases of the blood and blood-forming organs, inflammatory diseases, immune diseases, including autoimmune diseases, diseases of the nervous system, diseases of the circulatory system, diseases of the respiratory system, diseases of the digestive system, diseases of the skin, diseases of the musculoskeletal system. In further embodiments, the disease or disorder is cancer, diabetes or epilepsy.

The methods of the invention can be carried out in vivo or in vitro. Preferably, the reporter genes are functional under normal physiological conditions, comprising, for example, a pH from between about 6.8 to about 7.5; a pH from between about 6.9 to about 7.4; a pH from between about 7.0 to about 7.3; a pH from between about 7.1 to about 7.3; or a pH of 7. In certain conditions, the reporter composition is less functional or the function degrades, for example, under ischemic or apoptotic conditions.

The methods described herein have a variety of uses, for example, but not limited to methods of diagnosis in a clinical setting or methods to evaluate therapy, i.e. a patient's response to a particular treatment regime. For example, the methods are particularly useful to noninvasively monitor treatment response to small molecule and cellular therapeutics.

In certain aspects of the invention, one or more reporters as described herein can be administered to a subject. Known methods for administering therapeutics and diagnostics can be used to administer the reporter genes for practicing the present invention.

### Magnetic Resonance Imaging System

The invention also features a magnetic resonance imaging system. Magnetic resonance imaging systems are known in the art and commercially available. In certain aspects, the magnetic resonance imaging system comprises an imaging apparatus configured to perform a CEST MR technique, and one or more reporter genes or reporter substrate genes as described herein.

### Kits

Kits are also provided herein. For example, in certain aspects, the invention features kits for MRI imaging comprising one or more reporter genes or reporter gene substrates as described herein, and instructions for use. In other aspects, the invention features kits for MRI imaging comprising one or more vectors expressing one or more reporter genes or reporter gene substrates as described herein, and instructions for use.

The vector may be an expression vector adapted to be expressed in a subject. Examples of vectors include, for example, pIRES2-EGFP or pEF1alpha Myc/HIS

The kits may also provide means for administering the reporter genes, i.e. the polypeptides or nucleotides, by, for example, syringes. The kits may also provide buffers, pharmaceutically suitable carriers and the like. The instructions may provide information, for example, regarding storage, use, subject selection, administration, etc.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

The present invention features polypeptide or protein based reporters, wherein the MRI contrast is generated by the protein itself. The present invention also features enzyme based reporters, wherein the contrast is generated by the substrate/product of an enzyme. The present invention is described by, but not limited to, the following examples.

### Example 1

### MRI Biosensor for Protein Kinase A Encoded by a Single Synthetic Gene Methods

The following experiments were carried out with the below-described methods.

### Compound Preparation

Peptides were synthesized by NeoBioSci (Cambridge, Mass.) and provided as lyophilized powder. All compounds were dissolved in 10 mM PBS (pH 1/4 7.4) at the indicated concentration.

### CEST-MRI

CEST MRI experiments were performed on a vertical 11.7T Bruker Avance system. Approximately 20 mL of each sample was loaded into a capillary tube and up to 20 samples were scanned at each session to minimize variability as described before (Liu G et al., High-throughput screening of chemical exchange saturation transfer MR contrast agents. Contrast Media Mol Imaging 2010; 5:162-17). A modified acquisition with refocused echoes (RARE) (pulse repetition time/echo time1/48000/9.4 ms, RARE factor1/416, 1 mm slice thickness, field of view 1/4 11×11 mm, matrix size 1/4 64×32, resolution 1/4 0.17×0.34 mm, and number of averages (NA) 1/4 1-2) including a magnetization transfer module (B1 1/4 5.0 mT/4000 ms) was used to acquire CEST weighted images from −5 to +5 ppm (step 1/4 0.2 ppm) around the water resonance (0 ppm). To account for inhomogeneity of the B₀ field, the absolute water resonant frequency shift was measured using a modified water saturation shift reference method (Kim M et al., Water saturation shift referencing (WASSR) for chemical exchange saturation transfer (CEST) experiments. Magn Reson Med 2009; 61:1441-1450), using the same parameters as in CEST imaging except pulse repetition time 1/4 1.5 s, saturation pulse 1/4 500 ms, B1 1/4 0.5 mT, and a sweep range from −2 to +2 ppm (step 1/4 0.1 ppm). Data processing was performed using custom-written scripts in MATLAB. After B0 correction for each voxel using the water saturation shift reference data, mean CEST-spectra were calculated from a region of interest (ROI) for each sample. The MTR asymmetry was quantified as MTRasym 1/4 (S^{-Δω}−S^{+Δω})/S⁰ (S^{-Δω} and S^{+Δω} are the water peak differentials due to saturation at the target frequency +ω and its negative −ω, respectively. S⁰ is the unsaturated signal). Using the difference in signal between saturation at the target frequency and its opposite (negative) frequency cancels effects due to a direct, nonexchange mediated saturation of water.

### Phosphorylation Assay

PKA and ATP were purchased from New England Biolabs (NEB, Ipswich, Mass.) and provided in solution buffer. The reactions were completed with a peptide concentration of 1 mM in 1×PKA buffer and 4 mM ATP. Samples were prepared on ice. Then, following the manufacturer's protocol, samples were reacted for the indicated time at 30° C. using a bench-top 51000 Thermal Cycler (BioRad, Hercules, Calif.) and then heat-shocked at 65° C. for 20 min to inactivate the enzyme. Following heat inactivation, samples were stored at 4° C. until further analysis.

### Plasmid Construction

Plasmids were generated by custom synthesis of a gene encoding eight tandem repeats of the base sequence LRRASLG, with a 6-histidine C-terminal tag, whose coding sequence is optimized for expression in Escherichia coli (E. coli) (Genscript, Piscataway, N.J.). This gene was then subcloned into the pET expression system using TOPO cloning (Invitrogen, Carlsbad, Calif.).

### Protein Expression

E. coli: BL21(DE3) chemically competent cells (Invitrogen) were transformed with pET-LRRASLG8-His6. After induction in Magic Media™ (Invitrogen) at 30° C. for 18 h, the total protein was extracted using Cell Lytic B reagent (Sigma, St. Louis, Mo.). Lysate buffer was exchanged by passage through a Zeba 7K MWCO spin column (Pierce, Rockford, Ill.) with PBS (pH 1/4 7.4) as the elution buffer.

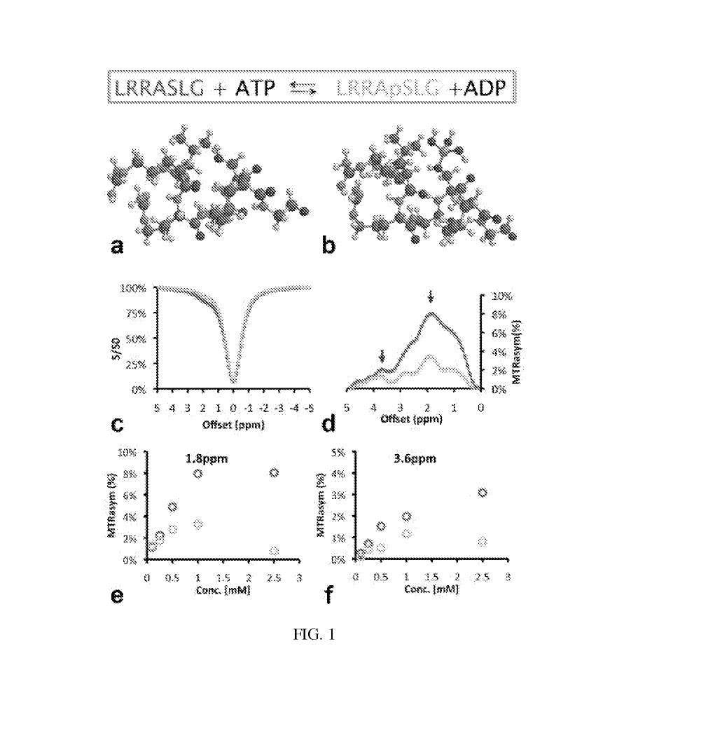

In the experiments described herein, CEST contrast was measured for the PKA consensus sequences LRRASLG (FIG. 1a) and its phosphorylated form, LRRApSLG, which has a phosphoryl group synthetically conjugated to the hydroxyl group of the serine, modeling the product of PKA action on the unphosphorylated substrate (FIG. 1b). In FIG. 1c, the CEST-spectra i.e., the normalized signal intensity of water protons is plotted as a function of the off-resonance saturation frequency for these two peptides at a concentration of 1 mM. For both peptides, the CEST spectra are asymmetric, indicating that these peptides generate CEST contrast, with the unphosphorylated form generating significantly higher contrast. There is a considerable difference in MTRasym between the unphosphorylated and the phosphorylated peptide, with two distinguishable peaks at 1.8 and 3.6 ppm (FIG. 1d) frequency offsets. This difference is directly concentration-dependent down to micromolar concentrations (FIG. 1e,f).

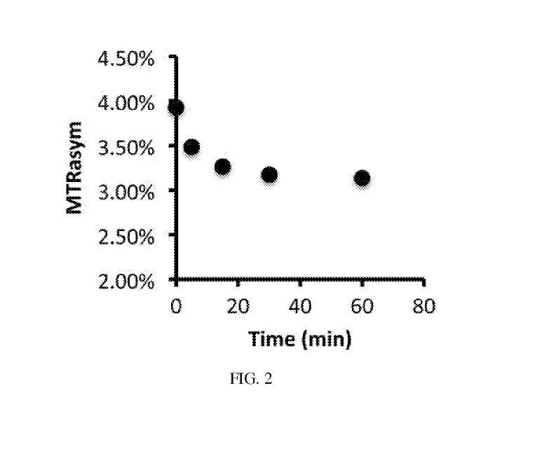

To test whether this effect can resolve the temporal action of PKA, recombinant PKA was incubated with the peptide LRRASLG for varying amounts of time (FIG. 2). A >50% decrease in CEST contrast was seen with kinetics similar to that expected for recombinant PKA activity, with the majority of the signal difference generated within the first 15 min of incubation.

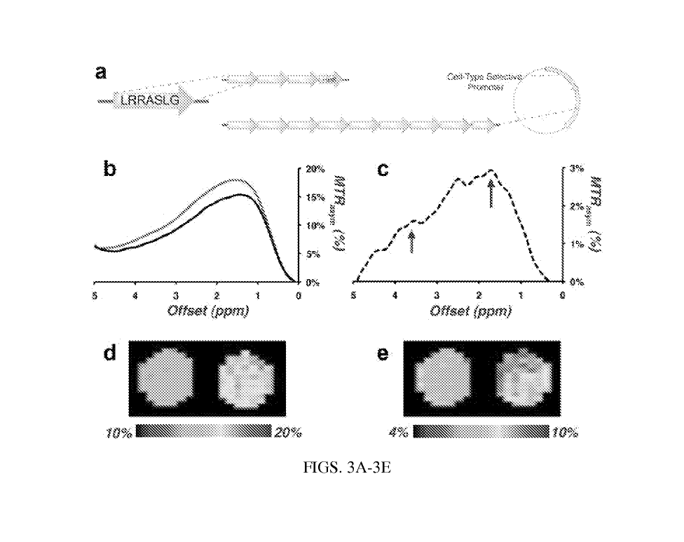

Next, an artificial gene that encodes eight tandems repeats of LRRASLG sequence was designed. The gene was cloned into a bacterial expression vector and was introduced into E. coli. FIG. 3b shows that a lysate of E. coli expressing the genetically encoded PKA biosensor provides higher MTR_{asym} compared to a lysate of E. coli expressing a control protein, cytosine deaminase, which shows no measurable CEST contrast of its own. This difference in MTRasym is more pronounced at the expected peaks of 1.8 and 3.6 ppm (FIG. 3c). Taken together, these results demonstrate the feasibility of detecting this genetically encoded candidate PKA biosensor in a biologically relevant environment.

The present invention sets forth a reporter gene substrate that is encoded by a single gene that may respond directly to PKA activity.

This reporter could potentially be further optimized using molecular tools, such as systematic mutagenesis, to improve the MRI sensitivity and the contrast between the phosphorylated and unphosphorylated state. Similar methods have been used to develop an MRI sensor for dopamine (Shapiro M G et al. Nat Biotechnol 2010; 28:264-270). This biosensor can be expressed in numerous target tissues using viral vectors for transduction (Iordanova B et al., Neuroimage 2012; 59:1004-1012) or in transgenic animals as was demonstrated with other genetically encoded MRI reporters (Cohen B et al., Nat Med 2007; 13:498-50331; Bartelle B B et al., Circ Res 2012; 110:938-947).

In this study, the arginine and serine content of consensus recognition sequences of proteins phosphorylated by PKA has been capitalized on. Both of these residues are expected to provide high CEST contrast (McMahon M T et al., Magn Reson Med 2008; 60:803-812). In particular, peptides with basic, positively charged lysine and arginine residues show high CEST contrast as the exchange rate of amide protons (NH) and amine/guanidyl protons (—NH3 [symbol] /1/4NH2 [symbol] ) is sufficiently fast to generate contrast, without being too fast to merge their resonance peak with that of water. Hydroxyl protons (—OH) of polar serine and threonine residues can also contribute to CEST signal and perhaps enhance contrast by inducing water coordination with nearby basic residues. This suggests that phosphorylation by serine/threonine kinases could measurably alter the exchange rates of substrate protons by (1) altering the exchange of nearby guanidyl and amide protons through coordination by the negatively charged phosphate group and (2) removing the CEST contrast generating hydroxyl proton of the phosphorylated serine or threonine. Indeed, the peptide described in our experiments exhibited a high CEST contrast in the unphosphorylated state, that is quenched by >50% in the phosphorylated state Importantly, this effect is conserved across a range of submillimolar concentrations that are similar to the expected intracellular concentration of functional reporter gene expression.

As can be seen in FIGS. 1 and 2, the change in the CEST contrast displays both a linear and nonlinear range. Thus, it is likely that protocols for in vivo applications will have to include acquisition of baseline data prior to manipulation of PKA and are only quantitative in a linear range of concentrations. These in vitro studies show that the sensor responds to phosphorylation on a timescale of minutes (FIG. 2). This is the same timescale that is required for acquisition of the CEST data. However, in vivo, enzymes such as phosphatases can remove the phosphate group from the substrate, and may thus reduce the measured PKA activity before the end of the acquisition. In the future, faster CEST acquisition schemes on a time-scale of seconds should dramatically improve the temporal resolution. It also should be noted that the accuracy of in vivo quantification of CEST agents at chemical shifts close to water resonance frequency may be greatly influenced by B₀ and B₁inhomogeneity. Therefore, the same water saturation shift reference protocol used here can be used to correct for B₀ inhomogeneity for in vivo studies, as it is suitable for correcting inhomogeneities of up to 1000 Hz 3(Liu G et al., Magn Reson Med 2012; 67:1106-1113). The B₁ inhomogeneity should also be measured separately and corrected if needed as shown previously (Singh A et al., Magn Reson Med 2012).

### Example 2

### Mammalian Expression of a CEST Reporter Gene Based on Human protamine-1

Methods

The following experiments were carried out with the below-described methods.

CEST experiments were performed on an 11.7T Bruker Avance system, as previously described. A modified RARE (TR/TE=6000/9.4 ms), including a magnetization transfer module (B1=4.7 μT/4000 ms), was used to acquire CEST-weighted images. The absolute water resonant frequency shift was measured using a modified WASSR method, with the same parameters as in CEST imaging, except for TR=1.5 sec and a saturation pulse of B1=0.5 μT/250 ms, which was used for B0 correction for each voxel using MatLab. MTR asymmetry (MTRasym)=100×(S−Δω−S+Δω)/S0 was computed at different offsets, Δω. Human Embryonic Kidney cells (HEK293), were engineered to express the gene encoding to hPRM1 (NM_002761; 293^{hPRM1}). Non-expressing wild type cells (293^{wt}) were used as controls. The cells were lysed or encapsulated in alginate-based microcapsules using Ba²⁺ ions as the gelating cation (Barnett, B. P., et al. Nat Protoc 6, 1142-1151 (2011)).

The purpose of the present experiments was to optimize the transgene expression in mammalian cells and image the cells in three-dimensional culture. One goal of these investigations was to capitalize on the biocompatibility of a human protein to enhance the CEST contrast while increasing cellular tolerance and avoiding an immune response.

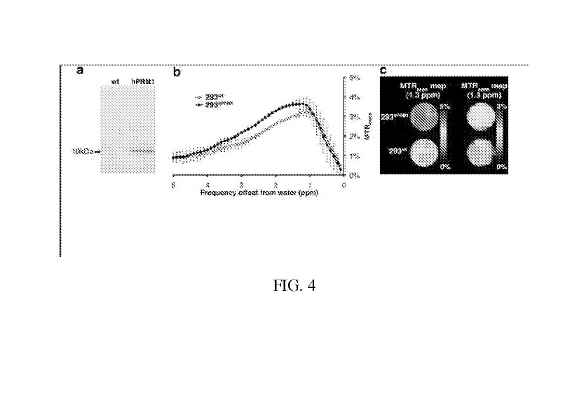

To examine the feasibility of detecting hPRM1 in mammalian cells using CEST, a lentivirus that encodes the hPRM1 under the cytomegalovirus (CMV) promoter was constructed and transduced into human embryonic kidney (HEK293) cells (FIG. 4a). A significantly higher CEST contrast was observed from the lysate containing the recombinant protamine. As can be seen in the MTR_{asym} plots (FIG. 4b), higher MTR_{asym} values were obtained for the 293^{hPRM1} lysate compared to the lysate from wild type cells. All the MTR_{asym} values from 1.3 ppm to 3.1 (19 points) showed a significant difference (p<0.05, student's t-test, unpaired two-tailed). At 3.6 ppm, the 293^{hPRM1} MTR_{asym} was still higher than the 293^{wt}; however, the p value was 0.098. This is likely due to the high variability. The MTR_{asym} maps (FIG. 4c), obtained following saturation at the guanidyl (1.3 ppm) and amide (3.6 ppm) resonance frequencies, demonstrated that indeed a higher CEST contrast was observed for the 293^{hPRM1}.

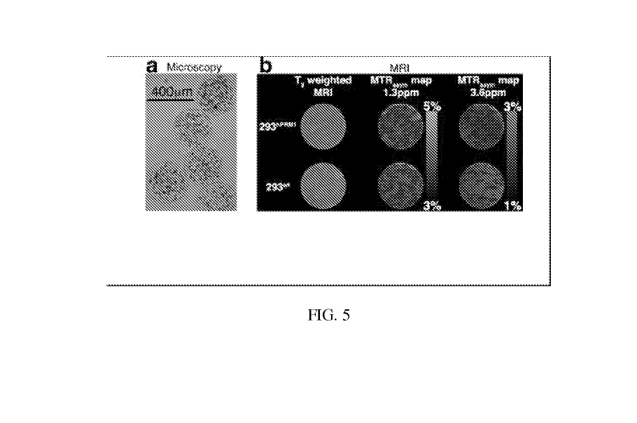

To demonstrate the ability to image hPRM1 expression in live cells using CEST, a three-dimensional culture of encapsulated 293^{wt} and 293^{hPRM1} cells was used. As expected, the CEST contrast from encapsulated live cells that express hPRM1 was higher than the contrast from non-expressing wild type cells. This observation was found for saturation at both 1.3 ppm and 3.6 ppm offsets from the water resonance frequency (FIG. 5).

The feasibility of using lysine- and arginine-rich synthetic genes as CEST based reporters has previously been demonstrated that hPRM1 over-expression could be detected in prokaryotic cells using CEST (Bar-Shir, A., et al. 2011). The present study has established that the human protamine could function as a CEST-based reporter gene in mammalian cells. In one of the many potential applications for hPRM1 in an immuno-isolation model of transplanted cells, cells are surrounded with thin alginate membranes that are permeable to soluble factors (e.g., insulin and metabolites), but are impermeable to native antibodies. This approach provides a means to reduce or avoid immunosuppressive therapy altogether. Although several methods have been successfully developed to monitor encapsulated cell transplantation (Barnett, B. P., et al. (2011); Arifin, D. R., et al. Radiology 260, 790-798 (2011), a non-invasive method to visualize the viability and function of the transplant at the molecular level remains an unmet need. Thus, developing a reporter gene, such as hPRM1, could be a most valuable tool.

The foregoing experiments illustrate the development of a novel MRI reporter gene based on human protamine-1 and demonstrated its expression and detection in eukaryotic cells, as well as in three-dimensional cell culture.

### Example 3

### Lysine Rich Green Fluorescent Proteins (LRgfP): Bi-Modal Reporter Genes for Monitoring Gene Expression with CEST and Optical Imaging

Methods

The following experiments were carried out with the below-described methods.

The E. coli optimized genes encoding to wild type GFP (wt) and its superpositive variants (+36 and +48), which were achieved by modified the solvent-exposed amino acids to lysine or arginine (M. S. Lawrence, et al., J Am Chem Soc 129, 10110 (2007), were obtained from Dr. David R. Liu (Harvard University, Cambridge, Mass.). The proteins were expressed in BL21 (DE3) E. coli and after induction in MAGIC MEDIA (Invitrogen) and purified using cobalt-based immobilized metal affinity chromatography. The expression and purity was determined by SDS-polyacrylamide gel electrophoresis (SDS-PAGE). Pure proteins were dialyzed against PBS and their CEST-MRI characteristics were measured as follows (G. Liu, et al. Contrast Media Mol Imaging 5, 162 (2010)): A modified RARE (TR/TE=6000/9.4 ms) including a magnetization transfer module (B₁=4.7 μT/4000 ms. The absolute water resonant frequency shift was measured using a modified WASSR method, with the same parameters as in CEST imaging except TR=1.5 sec and saturation pulse of B₁=0.5 μT/250 ms. Mean CEST-spectra were derived from a ROI for each sample, after B₀ correction for each voxel using MatLab. MTR asymmetry (MTR_{asym})=100×(S_{-Δω}−S_{+Δω})/S₀ was computed at different offsets Δω.

Positively charged amino acids (mostly lysine and arginine) in peptide and proteins make them attractive CEST-based contrast agents and reporter genes. The present experiments show that superpositively-charged mutants of the ultimate reporter gene green fluorescent protein (GFP) generate high CEST contrast. The water-exposed lysine and arginine amino acids of the mutants improve considerably the CEST contrast obtained from the guanidine (arginine) and amide (lysine and arginine) exchangeable protons, and thus, making these mutant GFPs bi-modal reporter genes for MR and optical imaging.

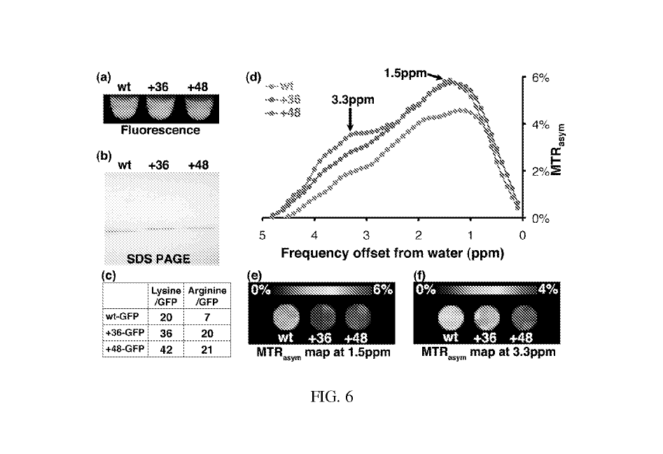

FIG. 6a shows fluorescence images of solutions containing the wt, +36 and +48 GFP variants. The high purity of the three proteins was validated by SDS-PAGE (FIG. 6b). The CEST-MRI characteristics of the three examined GFPs (wt, +36 and +48) were examined (FIG. 6d-f). Both superpositive GFPs (+36 and +48) generated higher MTR_{asym} values as compare to wt-GFP at both 1.5 ppm (guanidine protons of arginine) and 3.3 ppm (amide protons) frequency offsets from water. Moreover, the +48 GFP generated higher MTR_{asym} values at the amide frequencies (3.3 ppm) than the +36 mutant. FIG. 1e-f display MTR_{asym} maps that reveals correlation between the protein charge and the CEST contrast especially when a saturation pulse was applied at 3.3 ppm offset (FIG. 60.

Superpositively charged GFPs are highly resistant to aggregation, retain high fluorescence levels even after being boiled and cooled and reversibly complexed DNA and RNA (M. S. Lawrence, et al. (2007)). The present experiments demonstrate that in addition, the superpositively +36 and +48 GFPs generate high CEST-MRI contrast. These results are in a good agreement with previous reports showing that high content of the positively charged amino acids (either lysine or arginine, FIG. 6c) revealed in high CEST contras (G. Liu, et al. Contrast Media Mol Imaging 5, 162 (2010)). Both mutants (+36 and +48) contain equal number of arginine residues (20 and 21, respectively) and therefore generate the same MTR_{asym} value from the guanidine exchangeable protons at Δω=1.5 ppm (FIG. 6d-e). However, +48-GFP that have more lysines as compare to +36-GFP (42 and 36, respectively, FIG. 6c), thus, generates higher MTR_{asym} values at the resonance of the amide protons, i.e. at Δω=3.3 ppm (FIG. 6d,f) as expected from lysine rich proteins (Gilad et al., Nat Biotechnol 25, 217 (2007); G. Liu, et al. (2010)).

### Example 4

### Monitoring Enzyme Activity Using a Diamagnetic Chemical Exchange Saturation Transfer Magnetic Resonance Imaging Contrast Agent

Methods

The following experiments were carried out with the below-described methods.

Cloning and Expression in E. coli

The gene encoding the E. coli Cytosine Deaminase (CDase, CodA) was obtained from the American Type Culture Collection (ATCC Cat. 4099). The HSV1-tk gene was obtained from Dr. Martin Pomper (Johns Hopkins University). Both genes were sub-cloned into the pEXP5-CT expression vector (Invitrogen) in a reading frame with a C-terminal six-histidine tag under a T7 promoter. Both CDase and HSV1-tk were transformed into BL21 (DE3)-competent E. coli. The recombinant proteins were expressed after induction in Magic Media™ (Invitrogen), and both could be detected with Western blot using an anti-6-histidine tag antibody.

### Cell Culture

Human HEK293FT cells (Invitrogen) were cultured in Dulbecco's modified Eagle's medium (DMEM) (Invitrogen) containing 10% fetal bovine serum (FBS, Gibco) and 1% penicillin/streptomycin (Sigma). Rat glioma 9L cells were grown in RPMI medium (Sigma) containing 10% FBS (Gibco), 2 mM L-glutamine (Invitrogen), 1 mM sodium pyruvate, 1:100 amphotericin B solution (Sigma), 0.06 mg/mL gentamicin (QBI), and 175 nM 2-mercapto-ethanol (Sigma). C17.2 mouse neural stem cells, stably expressing LacZ (courtesy of Dr. Evan Snyder), were grown in DMEM supplemented with 10% FBS (Gibco), 5% horse serum (Gibco), 2 mM L-glutamine (Gibco), and 1% penicillin/streptomycin (Sigma) at 37° C. under 5% CO2.

### Expression in Mammalian Cells

The pLenti-6-V5/DEST (Invitrogen) was used for lentiviral transduction. The ViraPower™ Promoterless Lentiviral GATEWAY technology (Invitrogen) was used to construct the vector. The cytomegalovirus (CMV) promoter was used for continuative expression of the CD gene. HEK293FT, 9L, and C17.2 cell lines were transduced in culture.

### CEST MRI

All MR images were acquired with a 9.4T Bruker Avance system equipped with a 15 mm sawtooth RF coil. A modified RARE sequence (TR=6.0 sec, effective TE=43.2 ms, RARE factor=16, slice thickness=0.7 mm, FOV=14×14 mm, matrix size=128×64, resolution=0.11×0.22 mm², and NA=2) including a magnetization transfer (MT) module (one CW pulse, B=3.6 pT (150 Hz), 3 sec) was used to acquire CEST-weighted images from −6 ppm to 6 ppm (step=0.2 ppm) around the water resonance (0 ppm). The absolute water resonant frequency shift was measured using a Water Saturation Shift Reference (WASSR) (Kim, M. et al. Magnetic Resonance in Medicine 2009, 61, 1441) method modified (Liu, G. et al. Contrast Media Mol Imaging 2010, 5, 162) with Lorentzian analysis. The same parameters as in CEST imaging were used except TR=1.5 sec, tₛₐₜ₌₅₀₀ ms, B1=0.5 pT (21.3 Hz) and the saturation frequency swept from −1 ppm to 1 ppm (step=0.1 ppm). A short spoiling gradient (0.21 msec, 150 μT/m) was used at the end of the saturation pulses to remove residual signals. The acquisition time was 48 sec for each CEST-weighted image and 12 sec for each WASSR image, which made the total acquisition time (including a z-spectra and a WASSR) approximately 52 minutes.

### CEST MRI Data Processing

Data processing was performed using custom-written scripts in MATLAB (Mathworks, Waltham, Mass.). CEST spectra were calculated from the mean of an ROI placed over each sample after B0 correction of the contrast on a per voxel basis. The CEST signal was quantified using MTR_{asym} at specific offsets of interest (i.e., &ω=+2 ppm for cytosine or +2.4 ppm for 5FC) using the definition: MTR_{asym}=(S^{-˜ω}−S^{+″ω})/S0, where and ₛ[−˜ω, +Aω] is the water signal intensity in the presence of a saturation pulse at offsets ±Aω, and S0 is water signal intensity in the absence of a saturation pulse.

Estimating the Exchangeable Rate Constants ₍ₖₑₓ₎ of Cytosine at 2.0 ppm and 5FC at 2.4 ppm

The exchange rate of exchangeable protons with a frequency of 2 ppm for cytosine and 2.4 ppm for 5FC at pH=7.4 was measured using the modified Quantifying Exchange using Saturation Time (QUEST) method (McMahon, M. T. et al. Magn Reson Med 2006, 55, 836). In brief, the CEST contrast for samples containing 20 mM cytosine or 5FC (40 mM of amine protons) at pH 7.4 was measured with saturation delays of 0.5, 1, 1.5, 2, 3, 5, and 9.5 sec, using a saturation field strength of 5.9 μT (300 Hz) and a repetition time (TR) set to 10 sec, using the RARE imaging sequence described above. The calculated MTR_{asym} values were then fit using numerical solutions to the Bloch-McConnel equations with an exchange rate (kₑₓ). The water T1w and T2w were experimentally determined as approximately 4 sec and 1.6 sec, respectively, using the saturation recovery spin echo method and the CPMG multi-echo spin echo sequence, respectively. The fixed model parameters were water solute R1s=0.71 Hz and solute R2s=39 Hz.

### Validation of FLEX Experiments Using the FLEX Method

The exchange rates of the amine protons were quantified using the frequency-labeled exchange (FLEX) transfer principle (Friedman, J. I et al. J. Am Chem Soc 2010, 132, 1813). Briefly, the FLEX sequence uses a series of label transfer modules (LTMs) to selectively encode the chemical shift of exchangeable peaks as a function of offset frequency. Each LTM consists of a pair of selective 90x/90−x radiofrequency (RF) pulses, which encode the chemical shift evolution during an evolution time ₍t_{evol),} which is followed by an exchange time ₍t_{exch),} during which labeled protons exchange into the water pool. Each LTM is repeated multiple times to enhance the effect on the water pool. Analogous to CEST, the effect of exchangeable protons on the water pool can be observed by MRI measurements of the water signal intensity after a FLEX/CEST preparation period (tprep).

The FLEX data were acquired on a 9.4T Bruker Avance system equipped with a 10 mm sawtooth RF coil at 37° C. for 20 mM cytosine in PBS, The FLEX sequence consisted of 1000 LTMs followed by a spin-echo readout of the water signal intensity. Each LTM contained a pair of 0.22 ms block RF pulses with an offset frequency of 4400 Hz. _{texch} was set to 5 ms and ₜₑᵥₒₗ was varied between 0 and 4 ms in steps of 0.05 ms. All FLEX experiments were performed with a recycle delay between subsequent scans of 13 s (≧5×T1).

### In Vitro Detection of CDase Activity in Three Mammalian Cells

For each cell type, the same number of transduced (CD) or non-transduced (wild type, WT) cells were plated into 6-well plates (5.6×10⁶ HEK293FT cells, 10⁶ 9 L cells, and 1.4×10⁶ C17.2 cells, respectively). Approximately 6 hours later, fresh culture medium containing 7 mM cytosine or 10 mM 5FC was added to the cells. Fifty microliters of the culture media were collected at different time points up to 24 hours for HEK293FT cells (i.e. 0.5, 4, 6, and 24 hours), and up to 48 hours for the other two cell lines (i.e., 0.5, 4, 6, 24, and 48 hours). These samples were then transferred into capillaries and measured for CEST signal at 2 ppm for cytosine and 2.4 ppm for 5FC.

### ¹⁹F NMR Measurements

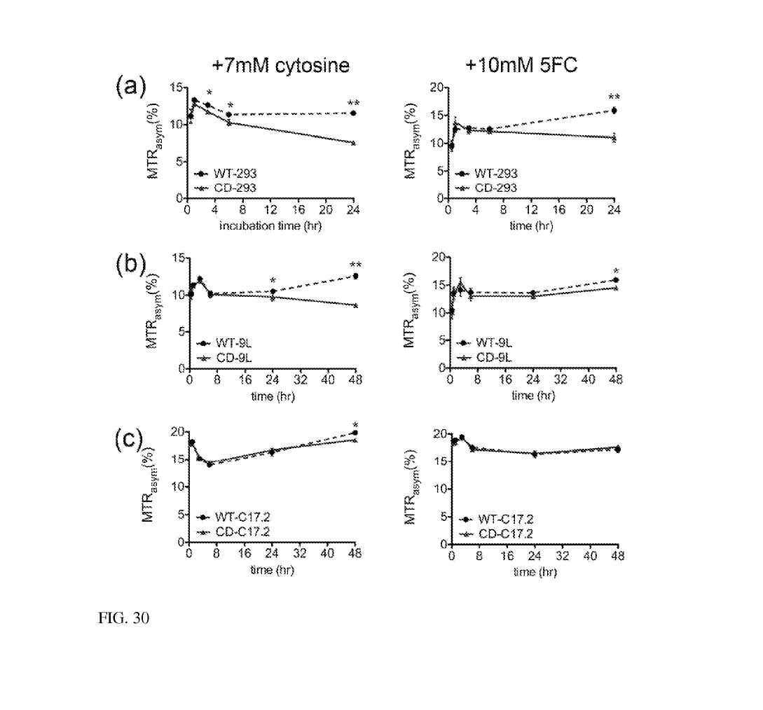

Studies were performed using a 500 MHz Bruker NMR spectrometer. ¹⁹F NMR spectra of the enzymatic mixture or aqueous solutions of 10 mM 5FC and 5FU standards (FIG. 30 a and b) were acquired using a single-pulse sequence with a flip angle of 90°, TR=5 sec, and NA=32, carried out at 25° C. with the spectra centered at the resonance frequency of 5FC. NMR data were processed and plotted using Topspin (Bruker, M A). For the cell culture media incubated with 10 mM 5FC for 24 and 48 hours for all three cell types, the ¹⁹F NMR spectrum was measured using the same protocol, except the number of averages was changed to 64 to improve SNR. 5FU peaks were only detected for HEK293FT cells after 24-hour and for 9L cells after 48 hours (FIG. 30 c,d). The complete results of the conversion detected using F NMR are listed in Table 1, as quantified by a conversion rate=peak area(5FU)/(peak area(5FU)+peak area(5FC)).

TABLE S1
¹⁹F NMR detection of conversion of 5FC to 5FU by three cell
types alter 24 or 48 hours incubation with 10 mM 5FC

| | HEK293FT | | 9L | | C17.2 | |
|---|---|---|---|---|---|---|
| | CD | WT | CD | WT | CD | WT |
| 24 hours | 38.9% | (−) | (−) | (−) | (−) | (−) |
| 48 hours | N/A | N/A | 8.5% | (−) | (−) | (−) |

N/A: not measured;
(−): no 5FU was detected.

### Cell Proliferation Assay

Both wild type- and CD-transduced cells were plated in 96-well cell culture plates to reach a sufficient cell number before 5FC treatment. Then media were replaced with media containing 10 mM 5FC. After 40 hours of incubation, cell viability was determined using a CCK6 assay kit (Invitrogen). CDase activity was validated by its functional ability to convert the pro-drug 5FC to active 5FU and induce a cytotoxic effect in cells. As seen in Figure S4, the two cell lines (HEK293FT and 9L) that showed detectable activity of CDase as measured with CEST MRI, exhibit a considerable decrease in the number of viable cells.

### Preparation of Cell-Containing Microcapsules

Unless otherwise stated, all reagents were used as received from Sigma, Avanti, and Invitrogen. Poly-L-Lysine (PLL) was purchased from Sigma-Aldrich and ultra-purified sodium alginate was purchased from FMC Biopolymers. Capsules were prepared using a one-step Lim-Sun method as previously reported (Lim, F. et al., Science 1980, 210, 908), except that an electrostatic (high voltage power supply) droplet generator was used to produce capsules with a smaller and more uniform size and higher strength, compared to that using an air-jet technique (Barnett, B. P. et al. Nat Protoc 2011, 6, 1142). In brief, a mixture of alginate and cells (1×10⁷ cells/mL) were passed through a needle at 100 ml/min using a nanoinjector pump. Droplets containing cells surrounded by the first layer of alginate were collected and transferred to a Petri dish containing 20 mM BaCl2 in 10 mM HEPES. The resultant gelled droplets were incubated with 0.1% PLL for 5 minutes for cross-linking alginate. The capsules were then washed and suspended in 0.15% alginate for another 5 minutes, followed by a final wash. The structure of microcapsules is shown in FIG. 115.

### Imaging of Microcapsules

Both WT-293 cell and CD-293 cells, at a density of 200-300 cells per capsule, were encapsulated one day before MR imaging. Approximately 100 capsules were transferred into 1 mm o.d. capillaries in 10 mM HEPES buffered saline (0.9% NaCl) at a final volume of 100 μL, which contained 5FC at a final concentration of 30 mM. The first CEST spectra acquisition, with frequencies swept from −3 ppm to +3 ppm (step 0.2 ppm), was performed at approximately 1 hour after incubation. Then CEST spectra imaging was repeated another 3 times. After the first and the last CEST spectra acquisition, two WASSR spectra were collected to correct the B0 inhomogeneity.

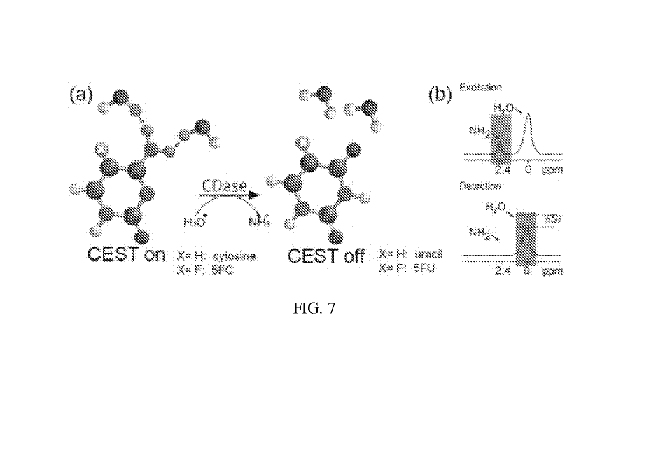

In the following experiments, the enzyme cytosine deaminase (CDase) is used to demonstrate the feasibility of using CEST MRI as a specific tool for noninvasive real-time imaging of enzyme activity using metal-free bio-organic diamagnetic substrates (DIACEST). CDase catalyzes the conversion of cytosine to uracil through the removal of an amine group, or “deamination”. It can also convert the prodrug 5-fluorocytosine (5FC) into the chemotherapeutic agent 5-fluorouracil (5FU), making it a promising enzyme/prodrug system for cancer therapy (Aghi et al., J. Gene Med. 2000, 2 148). Because CDase activity is absent in mammalian cells, the administration of 5FC is not likely to result in significant toxicity to normal tissue. Since amine groups contain two exchangeable protons, it was hypothesized that deamination of cytosine or 5FC by CDase to generate uracil or 5FU should be detectable by CEST MRI (FIG. 7).

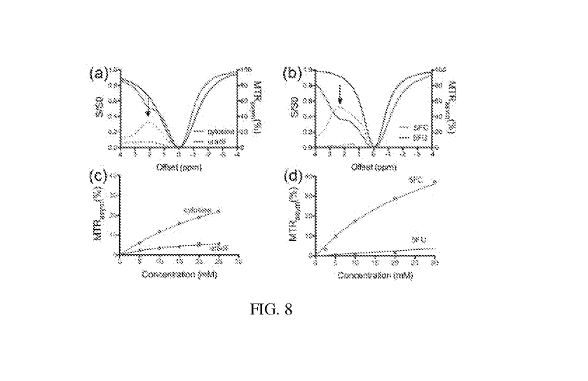

It was first determined whether CEST MRI could detect the CDase substrates and products with sufficient sensitivity under physiological conditions. The CEST contrast generated by cytosine, uracil, 5FC, and 5FU was examined over a range of concentrations at pH 7.4 and 37° C. The solid lines in FIG. 8 a,b represent the CEST spectra, in which the water proton signal is plotted as a function of saturation frequency. The dashed lines represent plots of MTRasym, a measure of CEST contrast defined by MTRasym=(S^{-Δω}−S^{Δω}/S0, in which S^{-Δω} and S^{Δω} are the MRI signal intensities after saturation at −Δω and +Δω, where Δω is the frequency offset from the water proton frequency (set at 0 ppm by convention), and S0 is the intensity in the absence of a saturation pulse. The maximal MTRasym values for cytosine and 5FC were obtained at offsets of +2 ppm and +2.4 ppm, respectively. In contrast, uracil and 5FU, which do not have an NH2 group, showed only limited and non-frequency-specific MTRasym that is probably due to the rapidly exchanging 1- or 3-imino NH protons, although this has not been confirmed. FIG. 8c,d shows the dynamic range of these substrate concentrations that can be detected with CEST MRI. These findings indicate that CEST MRI is suitable for monitoring the deamination of cytosine and 5FC.

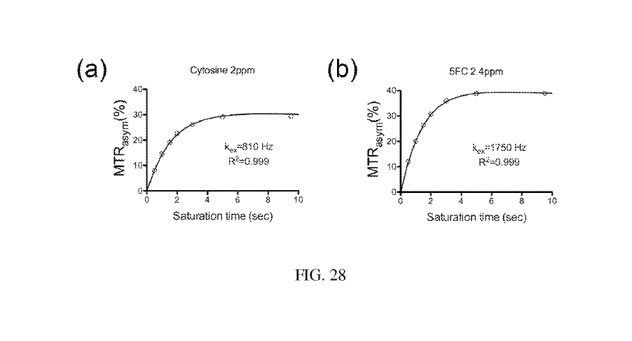

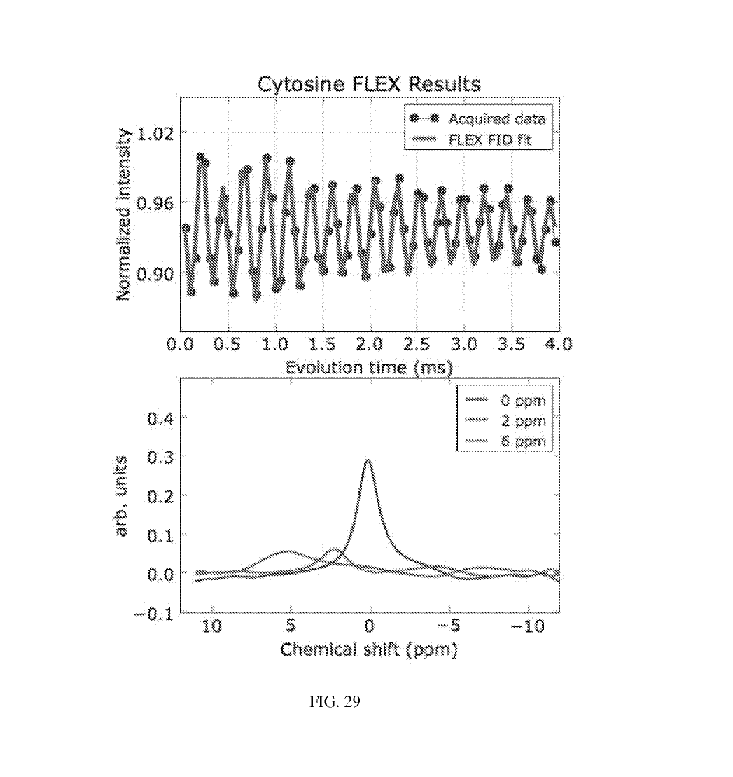

The exchange rate (kₑₓ) of the amine protons of cytosine was determined to be 8.1×10² Hz by measuring MTR_{asym} as a function of saturation time (McMahon et al C.Magn. Reson 2000, 143, 79) (FIG. 28). This was in good agreement with the value of 9.3×10² Hz determined using an alternative approach, the frequency-labeled exchange (FLEX) transfer method 17 (FIG. 29). For the amine proton of 5FC, kₑₓ was found to be 1.8×103 Hz (FIG. 28b). For both cytosine and 5FC, kₑₓ is on the order of magnitude of the frequency difference with water (Δω), in the intermediate exchange range. Notably, CEST can still detect such rapidly exchanging protons, such as OH groups, as long as some partial saturation can be achieved while the proton is at the correct frequency. This is an advantage over conventional MRI, where it would not be detectable.

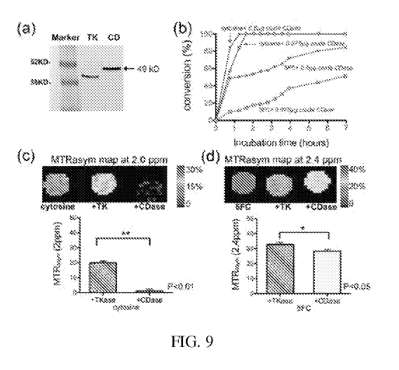

Next, the detection of recombinant CDase activity was evaluated with CEST MRI. The gene encoding the Escherichia coli CDase (CodA) was cloned into an expression vector (pEXP5-CT; Invitrogen) in a reading frame with a six-histidine C-terminal tag under the regulation of the T7 promoter. The gene encoding the herpes simplex virus type-1 thymidine kinase (HSV1-tk) was used as a control. Both enzymes were over-expressed in E. coli (BL21), and a crude protein extract was used to measure enzymatic activity with MRI. As shown in FIG. 9b, with recombinant CDase, substrate conversions could be measured for both cytosine and 5FC. FIG. 9c,d demonstrates that the reduction in MTRasym in the presence of CDase is significantly larger than that in the presence of the control enzyme HSV1-tk. While the conversion of cytosine was nearly complete after 24 h, this was not the case for 5FC. This can be attributed to the lower specificity of CDase toward 5FC relative to its natural substrate cytosine. A slight reduction in MTRasym was also observed in the extract containing recombinant HSV1-tk, possibly resulting from endogenous CDase activity.

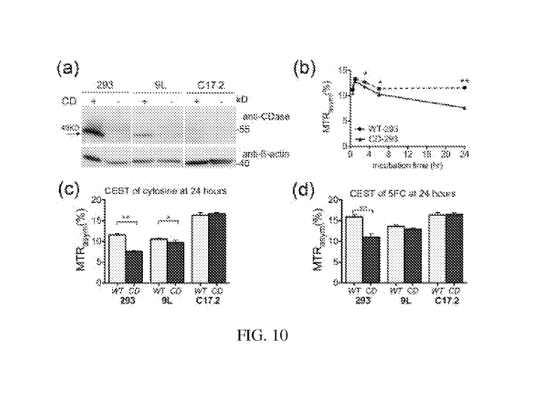

Therefore, the CDase activity in mammalian cells that do not express endogenous CDase was tested. A lentivirus that encodes CDase under the CMV promoter was constructed. The lentivirus was used to transduce three different cell lines in culture. As shown in FIG. 10a, the cell lines expressed different levels of CDase. Human embryonic kidney (HEK293FT) cells expressed the highest amount, 9L rat glioma expressed an intermediate level, and C17.2 mouse neural stem cells failed to express the enzyme at a detectable level.

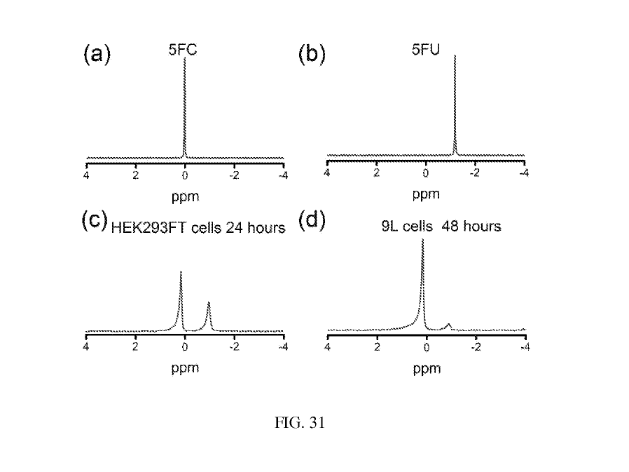

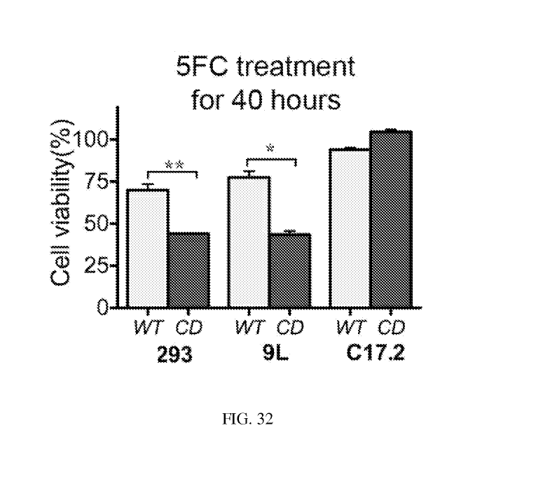

For each cell type, the same number of transduced or untransduced [wild-type (WT)] cells were plated (5.6×106 HEK293FT, 106 9L, and 1.4×106 C17.2 cells). Fresh culture medium containing 7 mM cytosine or 10 mM 5FC was added to the cells, and 50 μL of the culture medium was collected at different time points up to 48 h and measured with CEST MRI in capillaries, as described previously. FIG. 5b demonstrates a significant difference in MTR_{asym} for transduced and nontransduced HEK-293FT cells as early as 4 h after incubation with 7 mM cytosine. This is in good agreement with the high expression level of CDase by those cells. In contrast, for cells treated with 10 mM 5FC, a significant difference in MTR_{asym} was observed only after 24 h (FIG. 30). FIG. 10b shows an initial increase in MTR_{asym} at the initial time points. This may be attributed to small changes in the ratio of enzyme to substrate, which may be the result of a reduction in the volume of the solution due to the sampling methods or of evaporation of minute amounts of the medium over time. Alternatively, changes in the pH of the cell culture medium may affect MTR_{asym}. Nevertheless, the experimental results show a significant difference between CDase-expressing cells and the controls, indicating the ability to monitor enzyme activity directly. FIG. 10c,d shows the difference in MTRasym for all cell lines after 24 h of incubation with cytosine and 5FC, respectively. At this time point, the HEK293FT cells showed a significant difference in MTRasym for both cytosine and 5FC. The 9L cells, which exhibited an intermediate expression level of CDase, showed a moderate reduction of MTRasym only for cytosine but not for 5FC. The C17.2 cells, which had undetectable CDase expression, showed no difference in MTRasym at this time point. (A complete time course of CEST MRI, validation with 19F-MR spectroscopy, and a cell viability assay can be found in FIGS. 30-32.

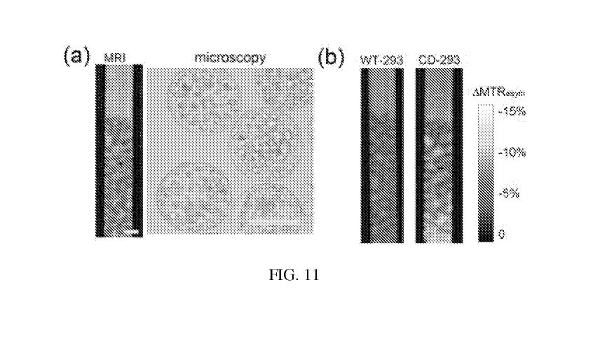

Immunoprotected cells have been evaluated as a new therapeutic alternative, for example, for pancreatic islet cell replacement in diabetic patients. In this approach, cells are encapsulated with alginate that enables passage of essential factors (e.g., nutrients and insulin) but protects cells from attack by the body's immune system. Several methods for monitoring encapsulated cells after transplantation have been successfully developed. Nevertheless, a noninvasive method for visualizing the viability of the transplant would be highly beneficial. In order to evaluate the feasibility of this method in cells, HEK293FT expressing CDase or nonexpressing control cells were encapsulated. As shown in FIG. 11, upon incubation with 5FC, only the CDase-expressing cells (CD-293) showed conversion of 5FC (initial concentration of 30 mM) within the first 3 h. On the basis of the cytotoxic mechanism of 5FU, it is unlikely that there is a contribution of cell death to the change in MTRasym. These findings indicate that CDase activity can be imaged at the macrocellular level in 3D culture. In addition, the CDase may be used not only to monitor the transplant viability but also as a suicide gene that can be used to eradicate transplanted cells in case of tumorigenic transformation of the transplanted cells.

These experiments demonstrate that the activity of the enzyme CDase can be monitored using CEST MRI. Previously, such detection was possible using 19F NMR spectroscopy and 19F NMR spectroscopic imaging, which rely on a change in chemical shift upon conversion of 5FC to 5FU. One advantage of using CEST MRI is that there is no need to use a toxic prodrug (5FC). Instead, the CDase's natural substrate, cytosine, can be used for imaging. This allows repetitive measurements and the use of CDase as a reporter gene. Another advantage is that CEST MRI can be used to monitor multiple enzymes simultaneously as long as their substrates have exchangeable protons that resonate at distinct frequencies. This property is relevant for studying gene networks, for example, in signal transduction cascade pathways.

An additional potential application may be real-time monitoring of the efficiency of therapeutic gene delivery and expression. As CDase has already entered the clinic for cancer gene therapy (Freytag et al. Cancer Res 2002, 62, 4968), such real-time monitoring may aid in predicting treatment outcomes. Since different cells or tumors may express differential levels of CDase, different patients may respond differently. Hence, real-time monitoring of enzymatic activity by CEST MRI could guide personalized medicine. Nevertheless, before this method can be fully translated, several hurdles must be overcome. Among these is optimization of the sensitivity of the substrates. The sensitivity in vivo depends on the expression levels of the enzyme, the cell density, and accessibility of the substrate to the CDase-expressing cells. The in vitro data indicate that recombinant CDase from 10⁶ mammalian cells is sufficient to reduce MTRasym significantly upon incubation with 7 mM cytosine or 10 mM 5FC. This number of cells is on the same order of magnitude as that used in cell-mediated CDase cancer gene therapy in vivo studies. The signal-to-noise ratio (SNR) of CEST MRI is 80, which is less than the SNR of 160 for 19F NMR (Figures S3 and S4 and Table S1). However, for the same level of CDase expressed by 9L cells, a relative change in MTRasym of 12% was observed after 48 h incubation with 5FC, compared with an 8.5% change measured using 19F NMR. With cytosine as the substrate under the same conditions, a much higher change (i.e., 50%) was observed with CEST MRI. Thus, the sensitivity of CEST MRI is comparable to that of conventional 19F NMR (Abogaye et al. Z. Cancer Res. 1998, 58, 4075).

A change of >5% in MTRasym was detected from 200-300 cells encapsulated in each 400-500 μm diameter alginate bead. As a 3D multicellular spheroid with a diameter of 300 μm consists of 3900 cells, CEST MRI is expected to allow measurement of CDase activity even when only 5-10% of the cells express the enzyme. Taken together, these results show that the present CEST MRI approach is expected to be sufficiently sensitive for future preclinical or clinical applications.

In addition, CEST contrast is highly dependent on the exchange rate, which can be modified using chemical modifications. This would be aimed toward increasing MTR_{asym} of the amine protons as well as the imino protons. The latter can be achieved by reducing the exchange rate at physiological pH, which may produce CEST contrast at 5-6 ppm. This would allow reduction of the applied B1, thereby decreasing the background from endogenous magnetization transfer effects as well as from direct water saturation. Shortening the image acquisition time is also required for improving the temporal resolution, which would allow more accurate dynamic measurement of the enzyme activity. Moreover, the enzyme turnover rate for substrates can be improved using genetic manipulations. The turnover rate was significantly improved when the entire gene or just its active site was subjected to directed evolution. It is noteworthy that T2 exchange effects may cause CEST agents to behave as T2 agents, resulting in darkening of the image. However, this is not a problem for the current agents at the low concentrations used and the chemical shift difference for these DIACEST agents. Finally, the detection may be improved by controlling the levels of the agents, for example by sustained release.

In summary, these experiments have demonstrated that CEST MRI, a novel approach for producing contrast based on proton exchange, can be used for direct real-time monitoring of CDase activity.

### Example 5

### Transforming Thymidine into a Magnetic Resonance Imaging Probe for Monitoring Gene Expression

Methods

The following experiments were carried out with the below-described methods.

(i) simulations of CEST data; (ii) CEST-MRI at 3.0 T clinical MRI scanner; (iii) cell viability assay; (iv) cell uptake assay; and (v) terminal transferase nick-end-labeling (TUNEL) assay.

### Simulations of CEST Data

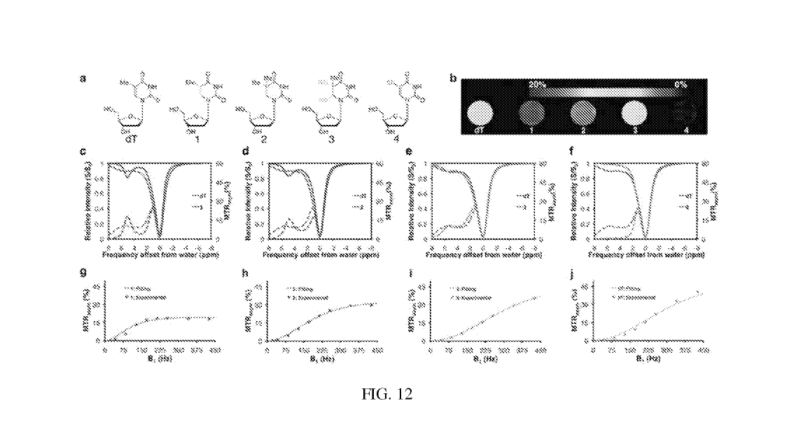

CEST data were simulated using the Bloch equations for a four-pool model (Woessner, D. E. et al. Magn. Reson. Med. 2005, 53, 790; Li, A. X. Magnetic resonance in medicine: official journal of the Society of Magnetic Resonance in Medicine) consisting of an imino NH proton, two hydroxyl protons, and bulk water protons for dT, 1, 2 and 3 (FIG. 12). Simulation parameters were determined from fitting the Bloch equations to CEST experimental data at 11.7 Tesla (Manuscript, FIG. 12 c-e). For convenience, other pools were simulated with the same relaxation parameters. Exchange parameters for each pool were determined from QUESP experiments (Manuscript, FIG. 12 g-j and Table 1). The simulations were performed for two different magnetic field strengths, 11.7 Tesla and 3.0 Tesla with an 8 s, 4 μT continuous wave (CW) CEST pulse.

### In Vitro Imaging Using a Clinical 3 Tesla MRI Scanner

Experiments were performed on a 3.0 Tesla Philips Achieva system (Philips Healthcare, Best, The Netherlands) using a body coil for transmit and a small animal solenoid coil for reception. CEST data were collected using a pulsed acquisition scheme (Jones, C. K. et al. Magn Reson Med 2011) with a 25 ms, 164 Hz frequency selective saturation pulse followed by a partial EPI readout (EPI factor 3). This TR interval was repeated until a 3D volume was acquired with 5 slices (slice thickness of 10 mm) across a field of view of 48×45 mm² (in plane resolution of 0.75 mm×0.9 mm) The acquisition per irradiation frequency consisted of TR/TE=65/5.5 ms and 4 averages. A total of 64 images were acquired which included an unsaturated volume and 63 saturated volumes with equally spaced frequency offsets between ±10 ppm (relative to the water frequency). The total acquisition time was 26 min 38 s. The experiments for B₀ correction (Kim, M. et al. Magn Reson Med 2009, 61, 1441) were run the same except that 40 Hz frequency selective saturation pulse was used. Data processing was performed as described herein.

### Cell Viability Assay

Cell viability assay was performed using CellTiter-Blue® Cell Viability Assay (Promega), which provides a fluorometric method for estimating the number of viable cells present in 96 well plate. HEK293FT cells transduced with the HSV1-tk (293^{HSV1-tk}) were plated in 96 well-plate (10,000 cells/well). Cells were incubated for a periods of 4 hours and 24 hours in the presence of ganciclovir (GCV, 0.5 mg/ml in cell medium), compound 2 (0.5 mg/ml in cell medium) and medium (as control). CellTiter-Blue® Reagent was added (20 μl/well) and plate was incubated for 3 hours at 37° C. Finally, fluorescence 560/590 nm was recorded and values obtained from probe containing wells (GCV or 2, N=4) were normalized to values of non-treated cells (control wells) to determine the % of viable cells after the incubation period.

### In Vitro Uptake Assay

One million cells (9L^{wt} and 9L^{HSV1-tk} in triplicates) were incubated in 200 ml medium containing 1 μCi [¹²⁵I]FIAU at 37° C. for 1 and 3 h. Cells were pelleted by centrifugation and washed 3 times with cold PBS. Gamma counter was used to monitor the radioactivity of the incubating medium and the incubated cells. [¹²⁵I]FIAU uptake was determined as the relative radioactivity (in percentage) of the cells to that of the incubating [¹²⁵I]FIAU medium (without cells).

### In Vivo CEST-MRI (Full CEST-Spectrum)

At the last time point of the experiment the full CEST-spectrum was acquired for each animal with the same parameters described in the Experimental Section (main text) except the followings: CEST-weighted images were acquired with a modified RARE pulse sequence (TR/TE=6000/35 ms), using a 213 Hz/4000 ms saturation pulse from −6 to +6 ppm around the water resonance, which was assigned to be at 0 ppm. Pixel-based B₀ correction was used as described before (Kim, M. et al. Magn Reson Med 2009, 61, 1441) using the same parameters as above except for TR=1500 ms, B₁/tₛₐₜ=21 Hz/500 ms, with a sweep range from −1 to +1 ppm (0.1 ppm steps). Mean CEST spectra were plotted from an ROI for each tumor and normal brain tissue, after B₀ correction. MTR_{asym}=100×(S^{-Δω}−S^{Δω})/S₀ was computed at different offsets Δω.

### Terminal Transferase Nick-End-Labeling (TUNEL)

ApopTag® Plus In Situ Apoptosis Fluorescein Detection Kit (Millipore) was used for detecting degraded DNA fragments to evaluate apoptosis after the administration of compound 2. Six hours after i.v. administration of 2 (150 mg/kg body weight) mice were transcardially perfused with 10 mM PBS and 4% PFA for tissue fixation. Brains were fixed in 4% PFA overnight, cryopreserved in 30% sucrose, and followed by cryo-sectioning at 30 μm slices. Slices were post-fixed in ethanol: acetic acid (2:1) solution followed by the manufacture procedure for using the ApopTag® Plus In Situ Apoptosis Fluorescein Detection Kit (Millipore, S7111).

### CEST Imaging Probes

5-Methyl-5,6-dihydrothymidine (2) and thymidine glycol (3) were prepared as previously described. 15,16 Thymidine (dT) and 5-chloro-2′-deoxyuridine (4) were purchased from Sigma-Aldrich, and (5S)-5,6-dihydrothymidine (1) was purchased from Berry & Associates, Inc.

### Cloning

The HSV1-tk gene was cloned into pEXP-5-CT (Invitrogen) for expression in Escherichia coli (pEXP-5-CT-HSV1-tk). For mammalian expression, the gene was cloned into pcDNA3.1 (Invitrogen; pcDNA3.1-HSV1-tk) and the pLenti-6-V5/DEST vector (Invitrogen; pLenti-6-HSV1-tk), both under a cytomegalovirus (CMV) promoter and a fused V5-tag.

HSV1-TK Expression and Purification

BL21 (DE3) (Invitrogen) cells were transformed with pEXP-5-CT-HSV1-tk. After induction in Magic Media (Invitrogen) at 30° C. for 18 h, the total protein was extracted, and the recombinant HSV1-TK protein fused to the six-histidine tag was purified using cobalt-based immobilized metal affinity chromatography (HisPur cobalt resin, Thermo Scientific). The expression and purity were determined by sodium dodecyl sulfate polyacrylamide gel electrophoresis (SDS-PAGE) and was validated by western blot using an anti-His antibody (Invitrogen).

### Expression in Mammalian Cells

Viruses were propagated in human embryonic kidney 293 cells (HEK-293FT). Forty eight hours after transduction, cells were lysed using Mammalian Protein Extraction Reagent (Thermo Scientific Inc.), and anti-V5 antibody (Invitrogen) was used for immunofluorescence and western blot analyses.

### pKa Values

The pKa values of the imino protons for the examined molecules were calculated using ChemAxon MarvinSketch v.5.3.3 software (www.chemaxon.com/marvin).

### Kinase Assay

In a solid white 96-well plate, 5 μM substrate (dT, compounds 1, 2, or 3; N=4 for each substrate) and 20 μM ATP were dissolved in 48 μL of kinase reaction buffer (40 mM Tris; pH=7.5, 20 mM MgCl2, and 0.1 mg/mL BSA) followed by the addition of 2 μL of purified HSV1-TK to initiate the kinase reaction. Eight wells without substrate were included as the control reference. The plate was incubated at room temperature for 2 h, after which 50 μL of Kinase-Glo luciferase reagent (Kinase-Glo max luminescent kinase assay, Promega Inc.) was added. After 30 mM, the resulting luminescence values were recorded and used to quantify the residual ATP in the reaction. The relative phosphorylation (%) was then defined as the luminescence signal obtained after the enzymatic reaction (substrate+HSV1-TK+ATP) relative to the signal obtained from wells without substrate (HSV1-TK+ATP). For a phosphorylation assay performed on HEK-293FT lysates, 2 μL of cell extract (either ^{293HSV1-tk} or 293^{wt} cells) was used instead of purified HSV1-TK, and the assay was performed using 1 or 2 as the substrate according to the procedure described above.

### In Vitro CEST-MRI

All compounds were dissolved at a concentration of 20 mM in phosphate-buffered saline (PBS) (pH=7.4) and placed in microcapillaries, as described previously.¹⁷ CESTMRI experiments were performed on a vertical 11.7 T scanner (Bruker Avance system) at 37° C. A modified RARE sequence (TR/TE=10 000/9.4 ms; RARE factor=16; 1 mm slice thickness; FOV=11×11 mm²; matrix size=64×32; resolution=0.17×0.34 mm²; and NA=2) including a magnetization transfer (MT) module (B1/tsat=170 Hz/4000 ms) was used to acquire CEST-weighted images from −8 to +8 ppm with increments of 0.2 ppm around the water resonance, which was assigned to be at 0 ppm. Pixel-based B0 correction was used as described before using the same parameters as above except for TR=1500 ms and B1/tsat=21 Hz/500 ms, with a sweep range from −2 to +2 ppm (0.1 ppm steps). Data processing was performed using custom-written scripts in MATLAB (Mathworks). Mean CEST spectra were plotted from an ROI for each sample, after B0 correction. MTRasym=100×(S^{-Aω}−S^{AωA}/^{‘-’}0)/3 was computed at different offsets Aω.

### Quantification of Exchange Rates

Exchange rates were quantified with the saturation power dependent CEST approach (QUESP) and Bloch equation fitting (McMahon et al., Magn. Res. Med. 2006, 55, 836). For each sample, the MTRasym was measured at the imino resonance frequency for CEST saturation fields of 43, 85, 128, 170, 213, 255, 340, and 426 Hz while the saturation pulse duration was kept constant at 4000 ms. The Bloch equations were then fit numerically to these _{MTRasym} values using MATLAB (Mathworks), and the exchange rates (kₑₓ, s⁻¹) were determined from the fits. Errors (95% confidence limits) were estimated using the F statistic.

### Cell Transfection and Transplantation

pcDNA3.1-HSV1-tk was used to transfect 9L rat glioma cells. A single clone was selected with 0.5 mg/mL G418 antibiotics. 9L^{HSV1-tk} and nontransfected wild type 9L cells (9L^{wt}) were inoculated (2×10⁵ cells/2 μL saline) bilaterally into the striatum of adult NOD-SCID male mice to generate intracranial 9L tumors in both hemispheres.

### In Vivo CEST-MRI

Data were acquired using a horizontal 11.7 T MRI scanner (Bruker, Biospec) equipped with a circular polarized MRI transceiver coil (i.d.=23 mm) Seven days after cell transplantation, mice were anesthetized with 1.5% isoflurane, and a series of four CEST data sets were obtained at 1, 2, and 3 h following intravenous (iv) injection of 2 in saline (150 mg/kg body). CEST weighted images were acquired with a modified RARE pulse sequence (TR/TE=6000/35 ms), using a 213 Hz/4000 ms saturation pulse alternating between ±5 ppm frequency offsets from the water frequency. For each time point, the MTR_{asym} map was calculated from four pairs of ₛ−5 ppm_{/s}+5 ppm using MATLAB (Mathworks). A single 1 mm slice with FOV of 1.6×1.6 cm² and a 128×48 matrix was used. To remove magnetization transfer effects, AMTRasym was defined as [MTR_{asym} (tumor)]−[MTR_{asym} (normal brain tissue)] as previously suggested.

### Immunofluorescent Histology

Mice were transcardially perfused with 10 mM PBS, followed by 4% paraformaldehyde (PFA) fixation. Brains were removed and fixed in 4% PFA overnight, cryopreserved in 30% sucrose, and followed by cryo-sectioning at 30 μm slices. Brain slices were blocked for 1 h at room temperature with PBS containing 5% bovine serum albumin (BSA), followed by overnight incubation in anti-V5 antibody solution. Nuclei were stained using 1 μg/mL DAPI (Invitrogen).

### SPECT/CT.

An X-SPECT small-animal SPECT/CT system (Gamma Medica-Ideas) was used for image acquisition as previously described (Nimmagadda et al., J. Nucl. Med. 2009, 50, 1124). Briefly, seven days after cell implantation, the mice were injected iv with 50.3 MBq (1.36 mCi) of ^{[125I]}FIAU. SPECT data were acquired 3 h after radiotracer injection, and the obtained images were coregistered with the corresponding 512 slice CT images. Fused SPECT/CT transaxial sections of 1 mm thickness were generated using Amira 5.2.0 software (Visage Imaging Inc.) showing the SPECT signal obtained from the brain.

In the experiments described herein, dT has been chemically modified to improve its CEST-MRI properties by increasing the imino proton pKa value and reducing its kex with water to comply with the slow exchange condition on the NMR time scale. The synthetic dT analogue 5-methyl-5,6-dihydrothymidine showed an excellent CEST contrast, as well as high specificity to HSV1-TK, and allowed successful in vivo monitoring of HSV1-TK expression in tumors with MRI.

### Dihydrothymidines Enhance the CEST Contrast In Vitro

Calculated and published pKₐ values of the imino proton of dT and four of its analogues (FIG. 12a) are summarized in Table 2, shown below.

| | dT | 1 | 2 | 3 | 4 |
|---|---|---|---|---|---|
| pK [symbol] ^{a} | 9.96 | 11.60 | 11.57 | 11.0 | 7.97 |
| pKₐ^{b} | 9.8²² | 11.6⁴³ | N.D | 10.7⁴⁴ | 7.9²³ |
| k [symbol] ^{c} (×10³ s⁻¹) | 5.1 ± 0.7 | 0.8 ± 0.2 | 1.7 ± 0.3 | 3.8 ± 0.2 | N.D.^{e} |
| MTR_{asym} (8.5 Hz) | 2% | 6% | 4% | 3% | N.D.^{e} |
| MTR_{asym} | 5% | 14% | 10% | 7% | N.D.^{e} |
| (128 Hz) | | | | | |
| MTR_{asym} | 10% | 18% | 16% | 11% | N.D.^{e} |
| (170 Hz) | | | | | |
| MTR_{asym} | 15% | 19% | 21% | 16% | N.D.^{e} |
| (213 Hz) | | | | | |

^{a}pK [symbol] values of the imino proton as calculated using MarvinSketch.
^{b}pK [symbol] values of the imino proton obtained from the literature.
^{c}Quantified exchange rates (×10³ s⁻¹) of imino and water protons. [symbol]
^{d}MTR_{asym} values (%) at 5 ppm for 20 nM agent.
^{e}N.D. = not determined. [symbol]
indicates data missing or illegible when filed

Upon hydrogenation of the 5,6-double bond of the pyrimidine ring, the pKₐ value of the exchangeable imino proton increases from about 9.8 for dT to 11.60 for 5,6-dihydrothymidine (1) due to the loss of the pyrimidine ring aromaticity. Substitution of the remaining hydrogen at position 5 in 1 with a methyl group produces 5-methyl-5,6-dihydrothymidine (2) and has a negligible effect on the pKₐ of its imino proton (pKₐ=11.57). The imino proton of thymidine glycol (3) has a pKₐ of 11.0, just between dT and compounds 1 and 2, due to its two additional hydroxyl electron-withdrawing groups at the pyrimidine ring. The substitution of the electron-donating methyl group of dT with an electron-withdrawing group, C1, to generate 5-chloro-2′-deoxyuridine (4), reversed the inductive properties of the 5-substituent and considerably reduced the pKₐ value of the imino proton to about 7.9 (Theruvathu, J. A. et al. Biochemistry 2009, 48, 11312).

Using an 11.7 T MRI scanner, the CEST contrast generated by solutions of these four deoxynucleoside analogues in PBS (pH=7.4, 37° C.) was compared with that of dT. The solid lines in FIG. 12c-f represent the CEST spectra, in which the water proton signal (S), normalized by the unsaturated signal (S₀), is plotted as a function of saturation frequency with respect to the water proton resonance frequency. This convention of assigning 0 ppm to the water protons is used in CEST-MRI and should not be confused with proton spectroscopy, in which the water resonance is assigned with respect to tetramethylsilane (TMS) or trimethylsilyl propanoic acid (TSP) and resonates around 4.7-4.8 ppm. The dashed lines represent plots of the asymmetry in the magnetization transfer ratio (MTR_{asym}), a measure of CEST contrast defined by the following: MTRasym=100%×(S^{-Δω}−S^{+Δω})/S₀, where S−Δω _{and} S^{+Δω} are the MRI signal intensities after saturation at −Δω and +Δω with respect to the water proton frequency. Taking the signal difference at −Δω and +Δω can remove confounding effects due to direct saturation of the water protons. The imino protons of compounds 1-3 showed a local maximum contrast at 5 ppm downfield of the water proton frequency, but only the dihydrothymidines (1 and 2) showed a well-defined sharp peak at that frequency (FIG. 12c-f).

Higher exchange rates increase the magnitude of the CEST contrast, which consequently improves the probe's sensitivity. However, if the exchange rate is too fast, the exchanging protons already exchange before being saturated by the CEST labeling pulse. Additionally, for fast exchange rates, the imino and water proton peaks can merge, and thus, the imino signal cannot be distinguished from the water signal. The maximal contrast (slow exchange regime) should be achievable by using an optimal saturation field (B₁) that can be predicted by the expression B₁(optimal)=kₑₓ/27r (Woessner, D. E. et al. Magn. Reson. Med. 2005, 53, 790).

To assess this, the kₑₓ in the newly designed probes was quantified from the saturation power dependencies of the _{MTRasym} values (McMahon, M. T. et al. Magn. Reson. Med. 2006, 55, 836) at 5 ppm for 1-3 and at 6 ppm for dT (FIG. 12g-j, Table 1). The imino protons of the two dihydrothymidines, which have the highest pKₐ values (1 and 2; Table 1), best fulfill the “slow exchange regime” rule with kₑₓ values of 0.8±0.2×10³ and 1.7±0.3×10³ s⁻¹, respectively, for a chemical shift of Δw=5 ppm (=1.6×10⁴ rad/s at 11.7 T). The calculated optimal saturation fields (B₁) were 127 Hz for 1 and 270 Hz for 2. In fact, the MTRasym values of both compounds 1 and 2 reach a plateau at higher B₁ (FIG. 12g,h), when saturation fields increase above the optimal value. The imino proton of compound 3 is approaching the intermediate exchange rate range (kex=3.7±0.2×10³ s⁻¹) and therefore, shows a broadening of the MTR_{asym} curve compared to 1 or 2 (FIG. 12c-e). However, as predicted (Woessner, D. E. et al. Magn. Reson. Med. 2005, 53, 790) it produces a higher MTR_{asym} if a higher B₁ is used (FIG. 12i). No CEST contrast was observed from the imino proton of compound 4 (Table 1, FIG. 12b,f), which has the lowest pKₐ value (7.97) and falls in the fast exchange regime (kex>>Δw) on the NMR time scale.

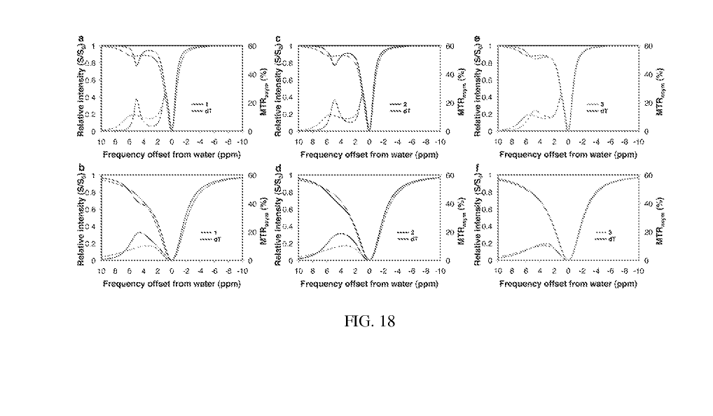

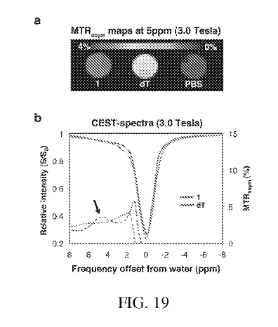

The results in FIG. 12 were obtained using an MRI scanner operating at field strength of 11.7 T. To evaluate the feasibility of imaging dihydrothymidines at lower field strengths typically used in clinical MRI, CEST experiments at 3 T were simulated (for dT and compounds 1-3) and performed (dT and 1). The results were compared to simulations and experimental data at 11.7 T. Compounds 1 and 2 fulfill the kₑₓ<Δω condition at both 11.7 and 3.0 T and showed a distinct peak at the 5 ppm offset from the water frequency for the two magnetic field strengths (FIG. 18). In contrast, for 3 and dT, which have a faster exchanging imino proton (5.1±0.7×10³ and 3.7±0.2×10³ s⁻¹, respectively, Table 1), no peak could be distinguished at the simulated CEST data for the lower magnetic field (FIGS. 18d and f), where the kₑₓ<Δω condition is not fulfilled for B₀=3.0 T. As mentioned, 1 fulfills the kₑₓ<Δω condition at 3.0 T and thus produces a visible peak at the 5 ppm offset from the water frequency at the CEST experimental data (FIG. 19b), as has been predicted by the simulations (FIG. 18b). In contrast, for dT, which has a faster exchanging imino proton, no peak could be distinguished and the MTR_{asym} value at 5 ppm obtained for 1 was 40% higher compared to dT (FIG. 19a).

### Dihydrothymidines are Phosphorylated by HSV1-TK

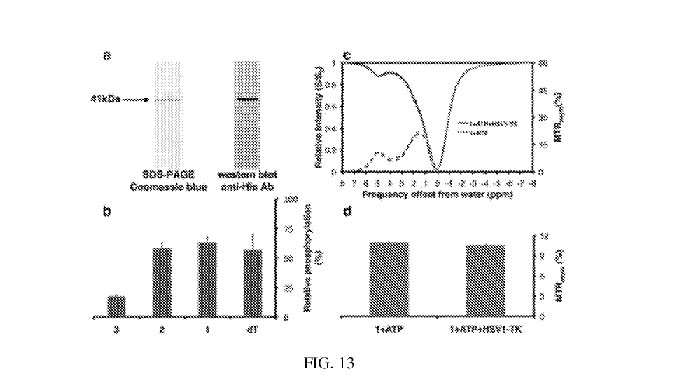

A major obstacle in designing new imaging probes is to maintain functionality after chemical modification. The phosphorylation level of each compound was examined by recombinantly purified HSV1-TK. The HSV1-tk gene was subcloned into the pEXP5-CT expression vector in a reading frame with a C-terminal six-histidine tag and subsequently transformed into BL21 (DE3) chemically competent E. coli. The recombinant HSV1-TK enzyme was expressed and purified using cobalt-based immobilized metal affinity chromatography (FIG. 13a). Notably, the observed phosphorylation of dT and analogues 1 and 2 by purified HSV1-TK was similar (>57%, FIG. 13b) after 2 h of incubation in the presence of ATP as the donor phosphate group. However, a lower degree of phosphorylation was observed for 3 (17%, FIG. 13b), which may be explained by the hydrophobic nature of the HSV1-TK nucleoside binding site.²⁹ Overall, these results demonstrate that 1 and 2, which yield CEST contrast that is superior to dT, can serve as CEST-based contrast agents for HSV1-TK, despite their chemical modification.

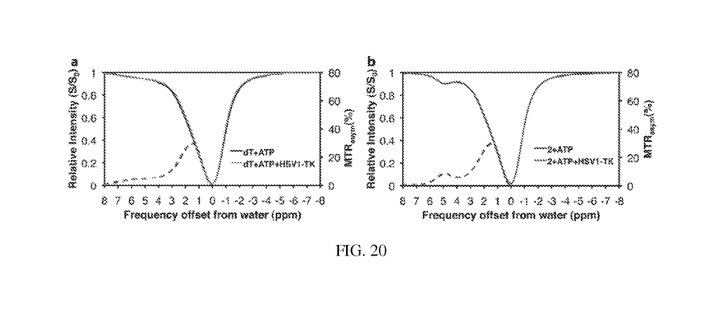

Next, whether the contrast is affected by phosphorylation was examined. CEST contrast was measured, with or without purified HSV1-TK, incubated in the presence of ATP for 2 h of incubation at 37° C. (pH=7.4), followed by 20 min of heat inactivation of the enzyme at 65° C. to end the kinase reaction. Since the phosphorylation site of the substrates is at the 5′-hydroxyl of the deoxyribose moiety, it is expected that the exchange rate of the imino proton and consequently the MTRasym value at 5 ppm would not be affected by phosphorylation. Indeed, the CEST contrast was not affected by phosphorylation, and the MTRasym values at 5 ppm were identical before and after the enzymatic reaction (FIG. 13c,d and FIG. 20).

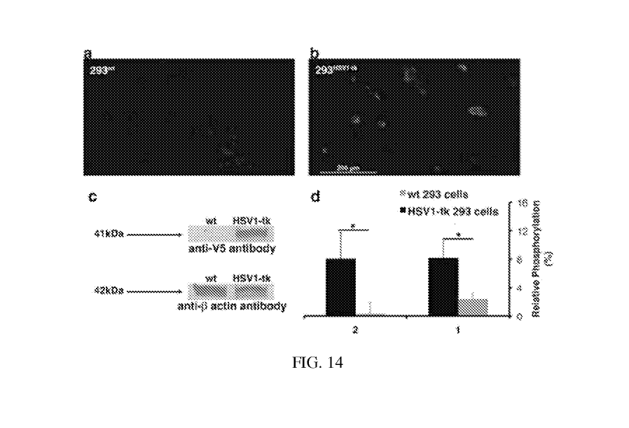

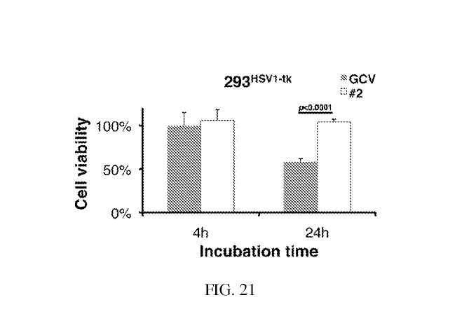

Additionally, the phosphorylation specificity of HSV1-TK was compared to endogenous mammalian thymidine kinases (mTK) for compounds 1 and 2. Toward that end, a lentivirus was constructed that encodes HSV1-TK under the CMV promoter and transduced HEK-293FT cells (FIG. 14a-c). The protein extracts of transduced 293^{HSV1-tk} cells (containing both HSV1-TK and m-TK) and wild type (293^{wt}) control cells (containing m-TK only) were used to measure enzymatic activity in the presence of either 1 or 2. FIG. 14d demonstrates that cell extracts containing HSV1-TK exhibited significantly higher phosphorylation of 1 and 2 than control cells. While compounds 1 and 2 had comparable phosphorylation rates, the increase in phosphorylation of 2 with respect to the control extract (293^{wt}) was greater by a factor of about 10 than that of 1, indicating that the additional methyl on position 5, which distinguishes 2 from 1, improves its specificity for HSV1-TK over m-TK. These results are in agreement with the findings that different dT analogues showed variable levels of phosphorylation by m-TK.²⁵ Moreover, it is important to note that such phosphorylation of 2 did not show any toxic effect on the 293^{HSV1-tk} (FIG. 21). This finding is in agreement with previous study showing that dihydrothymidines have no effect on DNA synthesis and do not increase the rate of mutations.²⁶ In contrast, the antiviral prodrug ganciclovir (GCV) resulted in a cytotoxic effect to the 293HSV1-tk upon phosphorylation (FIG. 21) as predicted and previously reported (Dhar, S. et al. Tissue Eng. 2007, 13, 2357).

Imaging HSV1-TK Activity In Vivo with 5-Methyl-5,6-Dihydrothymidine

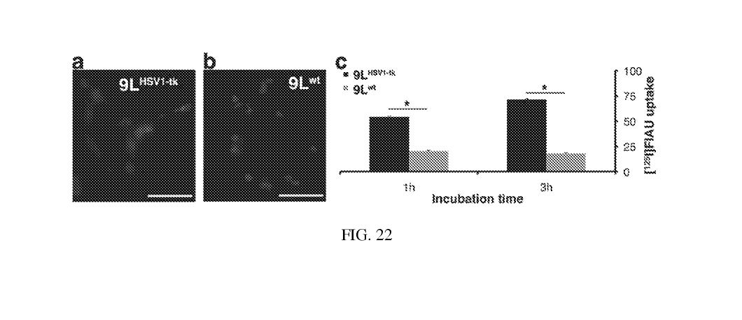

Next, the feasibility of using 2 as a CEST imaging probe for monitoring HSV1-TK in vivo was evaluated. The rat glioma cell line (9L) was transfected with the expression vector, pcDNA3.1, which encodes HSV1-tk under the regulation of the CMV promoter. Following antibiotic selection, a single clone that showed stable expression levels of the enzyme was selected (9L^{HSV1-tk}). Wild type, nontransfected 9L cells were used as controls (9L^{wt}). The HSV1-TK expression in 9L^{HSV1-tk} cells was confirmed by immunofluorescence (FIG. 22 a,b). The HSV1-TK activity was evaluated by an in vitro cell-uptake assay, in which a significant accumulation of the radioactive nucleoside [¹²⁵I]FIAU was observed in 9L^{HSV1-tk} cells after 1 and 3 h of incubation compared to 9L^{wt} cells (FIG. 22c).

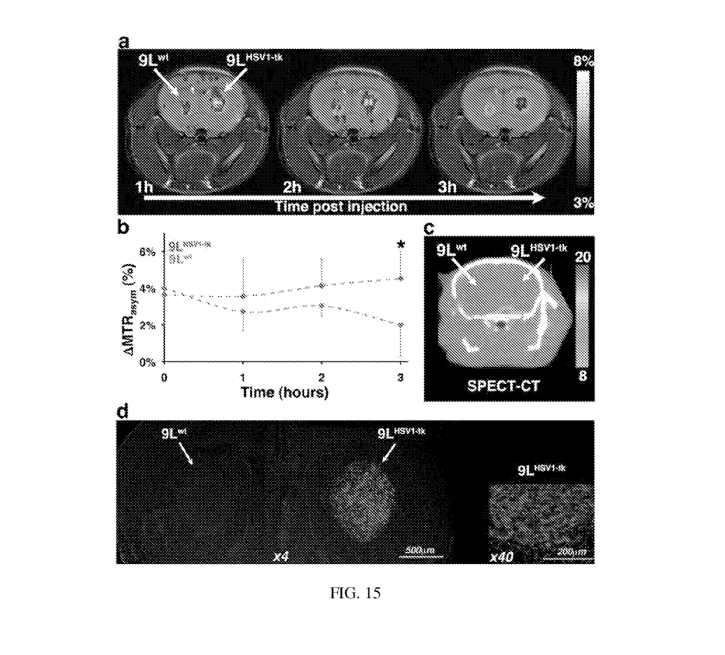

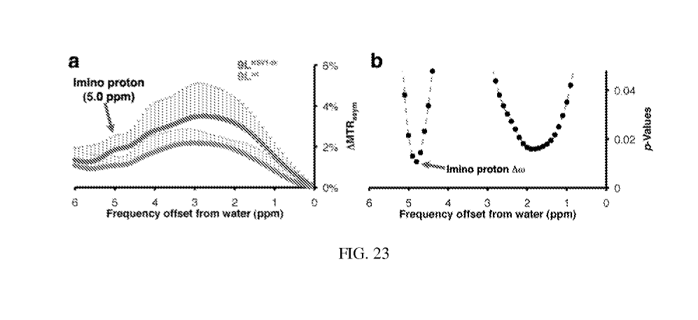

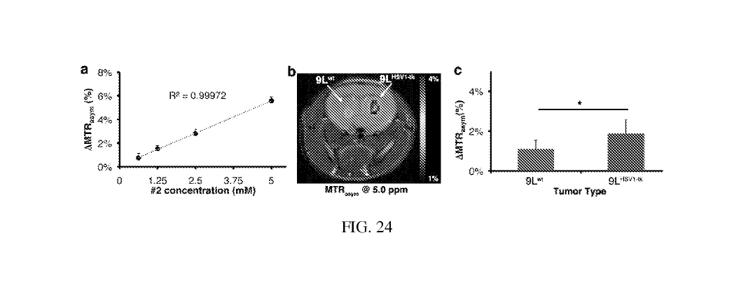

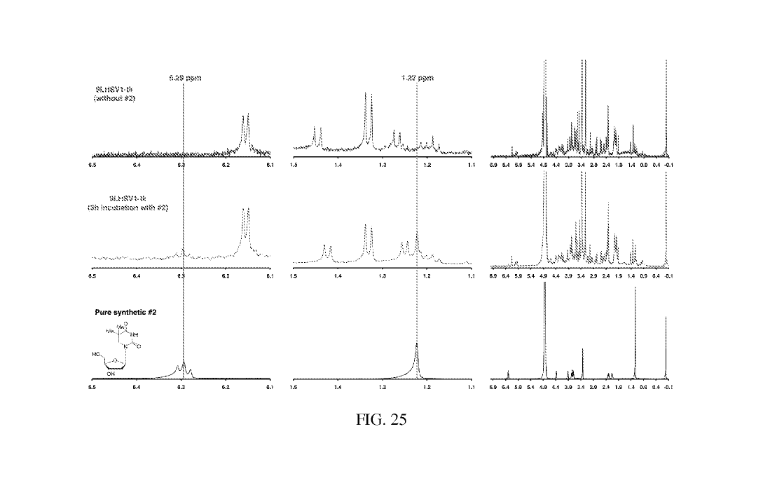

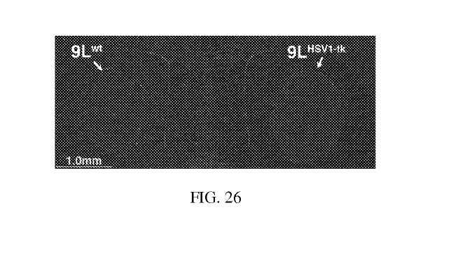

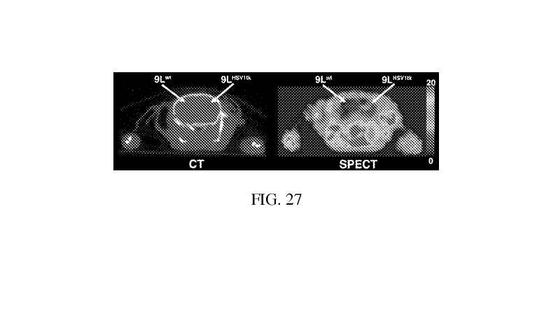

Inoculation of 9L^{HSV1-tk} to the ipsilateral striatum and 9L^{wt} into the contralateral striatum of adult male NOD-SCID mice (n=8) resulted in one tumor in each hemisphere of the brain. One week after tumor inoculation, analogue 2 was i.v. injected, and mice were scanned on a dedicated animal MRI scanner (11.7 T, horizontal bore). Maps of MTR_{asym} were acquired at 1, 2, and 3 h post-injection. FIG. 15a shows representative longitudinal MTR_{asym} (5 ppm) maps. As depicted in FIG. 15b, 3 h after injection of the probe, the normalized mean MTRasym values ₍ΔMTR_{asym)} from the 9L^{HSV1-tk} were significantly higher than those obtained from control tumors (4.5±1.5 for 9LHSV1-tk and 2.0±1.7 for 9L^{wt}; n=8; p=0.007). These findings indicate intracellular accumulation (approximately 0.7 mM, FIG. 23-25) in the 9LHSV1-tk tumors as opposed to clearance from control tumors. Such accumulation of 2 did not induce apoptosis neither in 9LHSV1-tk nor in 9L^{wt} as revealed by terminal deoxynucleotidyl transferase dUTP nick-end-labeling (TUNEL) staining (FIG. 26). The HSV1-TK functionality was confirmed by the administration of radiolabeled [¹²⁵I]FIAU using SPECT/CT. In agreement with the CEST-MRI results, accumulation of [¹²⁵I]FIAU was observed 3 h after iv injection only at the ^{9LHSV1-tk} tumor (FIG. 4c and FIG. 27). The high expression levels of the HSV1-TK in the ^{9LHSV1-tk} tumor was validated by immunofluorescence (FIG. 15d).

Overall, these data demonstrate that 2 can be used as an MRI probe to monitor HSV1-TK in vivo.

The experiments described herein show 2 as a functional probe for imaging HSV1-TK with CEST. Its exchangeable imino proton, which resonates at Δω=5 ppm, has a considerably lower kₑₓ than parent dT due to its higher pKa value allowing CEST detection with optimal MRI specificity. In addition, while this compound is phosphorylated by HSV1-TK, it is barely phosphorylated by endogenous kinases and therefore allows in vivo MRI of HSV1-tk gene expression with high functional specificity.

Due to the contribution of endogenous proteins and metabolites to in vivo CEST contrast following saturation in the Δω range 0-4 ppm from water (Cai, K et al. Nat. Med. 2012, 18, 302; Ling, W. et al. Proc. Natl. Acad. Sci. U.S.A. 2008, 105, 2266; van Zijl, P. C et al Proc. Natl. Acad. Sci. U.S.A. 2007, 104, 4359; Zhou, J. et al. Nat. Med. 2003, 9, 1085), it is imperative to design an imaging probe with an exchangeable proton that resonates downfield to this crowded endogenous range. The imino protons of the synthetic deoxynucleosides 1 and 2 not only resonate at Δω=5 ppm but also have a relatively slow kₑₓ compared to that of dT (Table 1) resulting in a well-defined peak centered at the CEST spectra. One advantage over previous generations of CEST reporter genes (Gilad, A. A et al. Nat. Biotechnol. 2007, 25, 217; Liu, G et al. J. Am. Chem. Soc. 2011, 133, 16326) is that the HSV1-TK CEST-based substrate provides a sharper signal at 5 ppm frequency offset. This makes 1 and 2 favorable DIACEST-based reporters and contrast agents due to less interference from contrast originating in other probes that have exchangeable protons resonating at different frequencies, for example, amide protons at Δω≈3.6 ppm (Zhou, J. et al. Nat. Med. 2003, 9, 1085; Gilad, A. A. et al.; Nat. Biotechnol. 2007, 25, 217), amine protons at Δω≈2-2.4 ppm (Cai, K. et al. Nat. Med. 2012, 18, 302; Liu, G. et al. J. Am. Chem. Soc. 2011, 133, 16326), guanidyl protons at Δω≈1.5-1.8 ppm (McMahon, M. T.; et al. Magn. Reson. Med. 2008, 60, 803), and hydroxyl protons at Δω≈0.9 ppm (Ling, W. et al. Proc. Natl. Acad. Sci. U.S.A. 2008, 105, 2266; van Zijl, P. C. Proc. Natl. Acad. Sci. U.S.A. 2007, 104, 4359). This may allow simultaneous imaging of multiple targets within the same sample (McMahon, M. T et al. Magn. Reson. Med. 2008, 60, 803) and therefore may play an instrumental role in studying complex biological systems. The reduced ₖₑₓ values of 1 and 2 allowed CEST detection not only at preclinical MRI scanners operating at high magnetic field (11.7 Tin this study) but also at a 3.0 T clinical scanner, where the condition kₑₓ, <Δω) is maintained. However, for clinical MRI scanners, specific absorption rate (SAR) and hardware limitations currently prevent optimal CEST-MRI measurements on low volume samples and small animals. These limitations were avoided in the 3 T experiments by using a pulsed CEST approach (Jones, C. K. et al. Magn. Reson. Med. 2012, 67, 1579); however, this technique produces less CEST contrast compared to conventional CEST methods. It is important to note that chemical exchange can affect the transverse relaxation as was measured with NMR in solutions containing diamagnetic proteins and metabolites (Rabenstein, D. L. et al. J. Magn. Reson. 1979, 36, 281; Zhong, J. H. et al. Magn. Reson. Med. 1989, 11, 295) and this property was used for measuring the exchange rate of protein in solution (Rabenstein, D. L. et al J. Magn. Reson. 1979, 36, 281; Zhong, J. H. et al. Magn. Reson. Med. 1989, 11, 295).

MRI has been used in the past for studying HSV1-tk as a therapeutic gene. The activity of HSV1-TK was measured as the outcome of treatment with GCV as was manifested by changes in T₂ and T1ᵣₕₒ° (Poptani, H. et al Cancer Gene Ther. 1998, 5, 101; Grohn, O. H; Cancer Res. 2003, 63, 7571). In those cases, the changes were observed within 4 and 2 days respectively. These results demonstrate that the HSV1-tk can be used as an MRI reporter gene for in vivo applications where the reporter activity is measured directly within hours. This is manifested by a statistically significant difference between the CEST contrast produced by 9L^{HSV1-tk} and 9L^{wt} tumors, 3 h after injection of compound 2. It is important to note that the kinetics of the imaging probe may vary between different biological systems or even among individual mice, for instance, due to the heterogeneity of the tumor vasculature, which may alter the accumulation and clearance of the imaging probe. In addition, in order to improve the sensitivity of the suggested system for monitoring enzyme expression, mutated HSV1-TK, which is adjusted to have a higher _{Vmax/Km} toward the imaging probe (2) and a lower Vₘₐₓ/Kₘ for dT, should be considered, in a manner similar to that applied for the PET imaging of HSV1-tk expression (Gambhir, S. S. et al. Proc. Natl. Acad. Sci. U.S.A. 2000, 97, 2785).

Since CEST contrast is visible only when a saturation pulse is applied, it may be used to evaluate multiple genes simultaneously using different MR reporter genes, including CEST reporter genes (Gilad, A. A. et al. Nat. Biotechnol. 2007, 25, 217; Liu, G. et al. J. Am. Chem. Soc. 2011, 133, 16326) paramagnetic MRI reporter genes that alter the transverse relaxation (T₂) of the tissue through accumulation of iron,^{37,38} or MRI-based reporter genes that are designed for either ³¹P- or ¹⁹F-MR spectroscopy. 39-41 Furthermore, with the recent development of combined PET/MRI high-field scanners ((Judenhofer M S et al., J. Nat. Med. 2008, 14, 459). Combining CEST with nuclear imaging could open up new avenues for multimodality molecular and cellular imaging.

In summary, the foregoing experiments demonstrate the identification of an imaging probe that provides frequency-specific MRI contrast with high functional specificity to HSV1-TK. Thus, it has been demonstrated, both in vitro and in vivo, the transformation of HSV1-tk into an MRI reporter gene. This imaging probe, which is stable for long periods, has the potential to allow serial monitoring of gene expression combined with high-resolution functional and anatomical MRI, as well as in conjunction with nuclear imaging. Overall, the principles outlined in this work may be further extended to a general paradigm for the design and synthesis of new imaging probes for in vivo imaging of a wide range of proteins en route to the elucidation of their biological function.

### Example 6

### Bioengineering a Reporter System that Combines a Highly Sensitive CEST and Fluorescence Probe

Methods

The following experiments were carried out with the below-described methods.

Pyrrolo-dC was dissolved in 10 mM PBS at 20 mM concentration. CEST MRI experiments were performed on an 11.7T Bruker Avance system as previously described (G. Liu et al., Contrast Media Mol Imaging 5, 162 (2010)). A modified RARE (TR/TE=6000/9.4 ms) including a magnetization transfer module (B1=4.7 μT/4000 ms) was used to acquire CEST weighted images. The absolute water resonant frequency shift was measured using a modified WASSR method, with the same parameters as in CEST imaging except TR=1.5 sec and saturation pulse of B1=0.5 μT/250 ms. Mean CEST-spectra were derived from a ROI for each sample, after B0 correction for each voxel using MatLab. MTR asymmetry (MTR_{asym})=100×(S^{-Δω}−S^{+Δω}/S₀ was computed at different offsets Δω. 9L rat glioma, engineered to express Drosophila melanogaster 2′-deoxynucleoside kinase (Dm-dNK; 9L^{Dm-dNK}) and control, non-expressing cells (9L^{wt}), 5×10⁶ cells per group, were incubated for 4 hours at cell-culture medium containing 2 mM pyrrolo-dC. Then, the cells were washed with PBS, lysed and fluorescence was measured in triplicates using a plate reader (lₑₓ355 nm/lₑₘ460 nm).

The Drosophila melanogaster 2′-deoxynucleoside kinase (Dm-dNK) enzyme phosphorylates a wide range of nucleoside analogs including the fluorescent nucleoside Pyrrolo-2′-deoxycytidine (pyrrolo-dC). It is shown here that the NH proton of the pyrrolo-dC generates high CEST contrast when saturation pulse is applied at 5.8 ppm from the water protons. The formation of the pyrrolo-dC monophosphate by recombinant Dm-dNK resulted in accumulation of the probe selectively in the cytoplasm of Dm-dNK expressing cells since its negative charge prevent cellular export. Hence, pyrrolo-dC can be used for monitoring the reporter gene Dm-dNK expression with CEST MRI.

The overall goal of the experiments described herein was to bioengineer a new MRI reporter system that allows real time monitoring of gene expression using CEST-MRI that encompasses optical capabilities. The specific goal of this study is to overcome challenges in quantifying the accumulation and sub-cellular distribution of the reporter probe, as well as to minimize contributions from endogenous CEST contrast or direct water saturation.

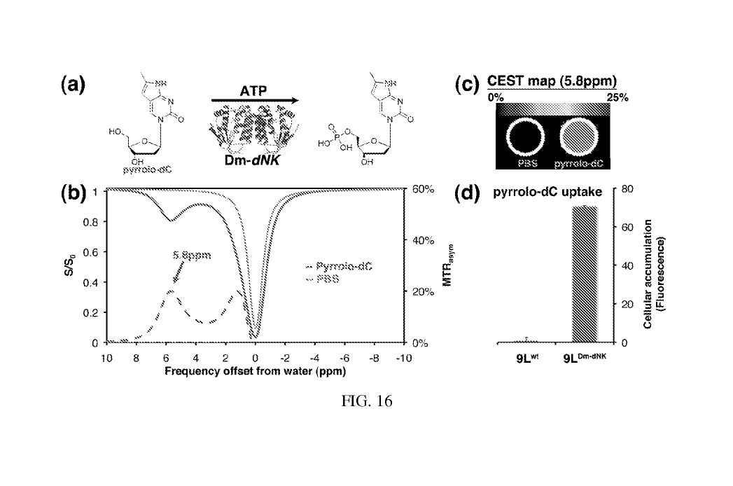

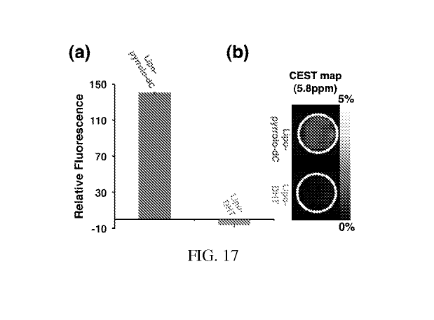

FIG. 16a shows schematic illustration of the phosphorylation of pyrrolo-dC to pyrrolo-dC monophosphate by the enzyme Dm-dNK in the presence of ATP. FIG. 61b shows the CEST-spectrum and MTR_{asym} plot of pyrrolo-dC (red) in comparison to those of PBS (gray). The NH proton of the pyrrolo-dC generate well-defined peak at 5.8 ppm frequency offset from water. The MTR_{asym} maps obtained at Dw=5.8 ppm (FIG. 16c) demonstrate the high CEST contrast generated by pyrrolo-dC. The optical properties to measure the accumulation of phosphorylated pyrrolo-dC in Dm-dNK expressing cells. The Dm-dNK gene was cloned into the pcDNA expression vector and was transfected into 9L rat glioma cells (9L^{Dm-dNK}). Next, both 9L^{Dm-dNK} and wild type (9L^{wt}) cells were incubated in a medium containing 2 mM pyrrolo-dC for 4 hours. As clearly shown in FIG. 16d only the lysate of incubated 9L^{Dm-dNK} cells provides high fluorescence level while no fluorescence was detected for incubated 9L^{wt}. As shown in FIG. 17a liposomes loaded with pyrrolo-dC showed higher fluorescence levels as compare to liposome loaded with the non-fluorescent nucleoside analog dihydrothymidine (DHT). The pyrrolo-dC containing lyposomes generate considerably high CEST contrast at 5.8 ppm as compare to DHT liposome (FIG. 17b)

For CEST-based application the observed sharp and better-defined NH peak at 5.8 ppm frequency offset from water is a major advantage, since a large Dw (>3.6 ppm) allows the detection of the compound with minimal contributions from endogenous CEST contrast and direct water saturation.

The formation of the pyrrolo-dC monophosphate in the presence of the recombinant Dm-dNK (FIG. 16a) resulted in accumulation of the fluorophor in the cytoplasm as its negative charge prevent cellular export. The use of fluorescent-based CEST-probe as pyrrolo-dC allows determination of its accumulation only in cells expressing Dm-dNK, which imply that in vivo monitoring of the Dm-dNK reporter gene expression with CEST MRI is feasible. To increase pyrrolo-dC circulation time, control its release and protect it from degradation in the circulation liposomes may be considered as a delivery vehicle.

Accordingly, the foregoing experiments demonstrate that synthetic fluorescent nucleosides such as pyrrolo-dC can be used for real time monitoring gene expression and tracking liposomal delivery with both optical and MRI modalities.

### Example 7

### Molecular CEST Imagining of Mucins with Different Glycosylation Levels

Deglycosylation of Mucin

Due to the complicated O-linked glycosylation of mucin, chemical deglycosylation is preferred over enzymatic methods (Edge, Biochem J. 376:2 (2003). The oligosaccharide chains on porcine stomach mucin (Sigma, M-2378) were removed using anhydrous trifluoromethanesulfonic acid (TFMS) treatment. Both deglycosylated and untreated mucin were dialyzed against water, lyophilized and dissolved at a conc. of 4.0 mg/ml in PBS (pH=7.1) for imaging.

### Encapsulated Cells

Three cell lines (MCF10A, non-tumorigenic human breast carcinoma; and LS174T and HT29, both human colon carcinomas) with different MUC-1 glycosylation levels were encapsulated in alginate-PLL-alginate microcapsules (Barnett et al., Nat. Prot. 6: (2011)) at 1000 cells/capsule in order to minimize cell sedimentation and variations in cell density.

### Image Acquisition and Analysis

Images were taken on a Bruker 11.7T scanner, using a RARE sequence with CW saturation pulse of B1=3.6 μT, Tₛₐₜ₌₃ s and frequency incremented every 0.2 ppm from −6 to 6 ppm for phantoms and every 0.25 ppm from −5 to 5 ppm for cells; TR=6 s, effective TE=17-19 ms, matrix size=96×64. CEST contrast was quantified by MTRasym=(S^{-Δω-}S^{+Δω})/S0 after a voxel-by-voxel B0 correction, with characterized mean Z-spectra and MTR_{asym} spectra for sample ROIs plotted.

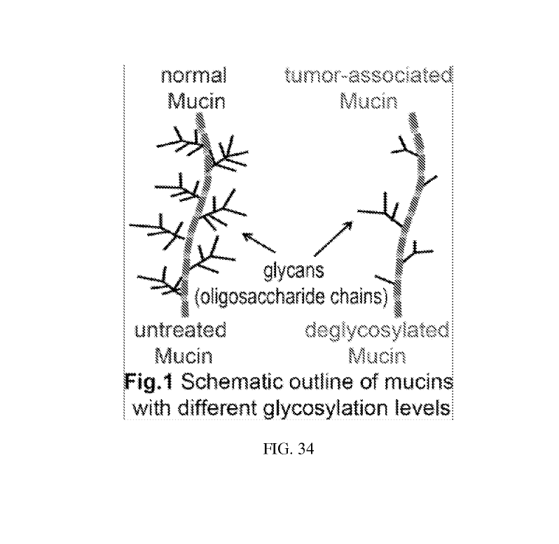

Tumor-associated glycosylation changes have been observed for decades (Hakomori, PNAS 99:16 (2002); Hollingsworth, Nat. Rev. 4: (2004)), and are associated with tumor proliferation, metastasis, and angiogenesis. Cell-surface glycoproteins including mucins, in particular MUC-1 (Hollingsworth, Nat. Rev. 4: (2004); Moore et al., Cancer Res. 64: (2004)) have been used as novel diagnostic and therapeutic targets. In many adenocarcinomas (e.g. colon, breast, and ovarian cancers), MUC-1 is overexpressed in aberrant forms generating an underglycosylated MUC-1 (uMUC-1) antigen (FIG. 34). Chemical Exchange Saturation Transfer (CEST) MRI is a molecular imaging modality that can amplify signals from specific functional groups in proteins, peptides, and sugars based on the exchange of their protons with water (van Zijl et al., PNAS. 104:(2007)). Owing to the abundance of both MUC-1 and its exchangeable protons on attached glycans (—OH) and core protein (—NH,—NH₂), the mucin shows a characteristic CEST contrast from 0.5 ppm to 4 ppm (Song et al, Proc.ISMRM 2334: (2012)). The present experiments are aimed at testing whether CEST MRI is able to detect changes in glycosylation levels of mucins, and to differentiate uMUC-1 positive tumor cells (expressing underglycosylated MUC-1) from uMUC-1 negative cells (expressing normally glycosylated MUC-1).

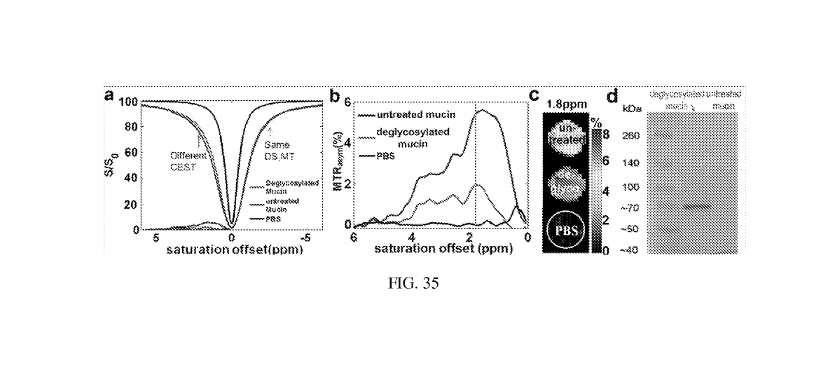

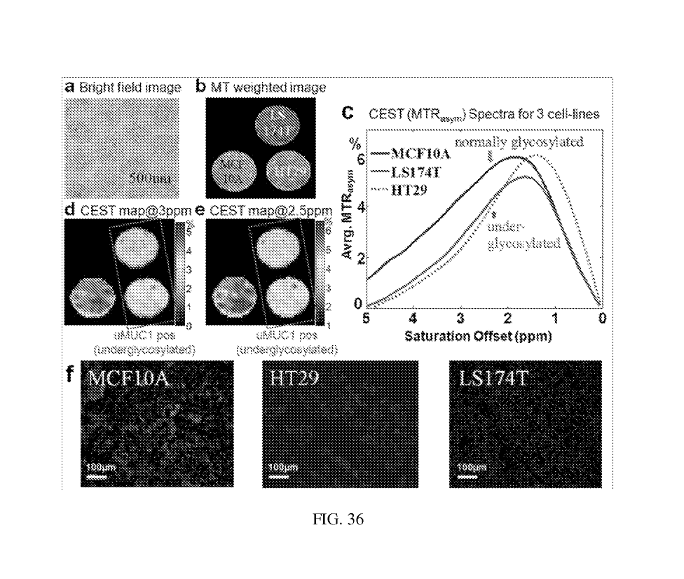

The untreated and deglycosylated mucin could be easily differentiated in both Z spectra and MTRasym spectra (FIG. 35a,b), with a significant reduction of CEST contrast over a broad chemical shift range, i.e. ˜80% reduction from 0.5 to 2 ppm and ˜50% loss from 2 to 4 ppm respectively. FIG. 35c is a MTR_{asym} contrast map at 1.8 ppm peak. The deglycosylation was confirmed by SDS PAGE electrophoresis (FIG. 35d), where deglycosylated mucin showed a MW of 70-100 kD, whereas untreated mucin did not show any bands due to the MW being >260 kD8. We then compared the CEST contrast produced by 3 cell lines with different MUC-1 expression: LS174T and HT29, both expressing underglycosylated MUC-1 (i.e., “uMUC-1 positive”), and MCF10A, expressing normally glycosylated MUC-1 (i.e. “uMUC-1 negative)”. Both the MTR_{asym} spectra (FIG. 36c) and contrast maps (FIG. 36d,e) clearly show that the underglycosylated MUC-1 tumor cell lines (LS174T and HT29) have a lower CEST contrast from 2 to 4 ppm. The MUC-1 glycosylation level was validated by immunostaining with an antibody detecting normally glycosylated MUC-1 (anti-MUC1 antibody, Epitomics) with red=MUC-1, blue=nuclei (DAPI) (FIG. 36f).

Deglycosylated and untreated mucin proteins could be easily differentiated by CEST MRI in vitro, with the deglycosylated sample showing >80% reduction in —OH peak. The MUC-1 cancer marker also exhibits differential CEST contrast between 0.5 and 4 ppm depending on the glycosylation levels, which is lower for the two cell lines having underglycosylated MUC-1. Our results suggest that CEST imaging of MUC-1 may potentially be used as a surrogate marker to non-invasively assess tumor malignancy and tumor progression.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

What is claimed is:

1. A method for obtaining an image by MRI, comprising:
introducing one or more genes into cells to be imaged, wherein the one or more genes are reporter genes or reporter substrate genes; and
imaging the cells using a Chemical Exchange Saturation Transfer (CEST) based MRI technique.

2. A method selected from the group consisting of
a method for real-time monitoring of gene expression by magnetic resonance imaging (MRI), comprising:
introducing one or more genes into cells to be imaged, wherein the genes are reporter genes or reporter substrate genes; and
imaging the cells using a CEST based MRI technique, wherein the imaging is performed in real-time;
a method for obtaining an image by MRI, comprising:
introducing one or more reporter genes into cells to be imaged, wherein the reporter genes encode one or more enzymes; and
imaging the cells using a CEST based MRI technique to detect substrate conversion by the enzyme; and
a method for real-time monitoring of gene expression by MRI, comprising:
introducing one or more reporter genes into cells to be imaged, wherein the reporter genes encode one or more enzymes; and
imaging the cells using a CEST based MRI technique to detect substrate conversion by the enzyme, wherein the imaging is performed in real-time.

3. The method of claim 1, wherein the cells are eukaryotic cells.

4. The method of claim 1, wherein the reporter gene encodes a human protein, optionally human protamine-1.

5. (canceled)

6. The method of claim 1, wherein the reporter gene encodes an E. coli protein.

7. The method of claim 6, wherein the protein is a fluorescent protein, optionally GFP or a superpositive GFP variant.

8-9. (canceled)

10. The method of claim 1, wherein the reporter substrate gene encodes a protein that responds to protein kinase activity, optionally a protein kinase selected from Protein Kinase A and AKT/Protein Kinase B.

11. (canceled)

12. The method of claim 1, wherein the reporter substrate gene encodes a protein sequence with one or more amino acid residues selected from the group consisting of: lysine, arginine, serine and threonine, optionally the reporter substrate gene encodes one or more tandem repeats of the sequence LRRASLG or the reporter substrate gene encodes one or more tandem repeats of the sequence RXRXX(S/T), wherein R=arginine, X=any amino acid, and Y=hydrophobic amino acid.

13-14. (canceled)

15. The method of claim 1, wherein the reporter gene or reporter substrate gene is mutated to improve MRI sensitivity.

16. The method of claim 1, wherein the reporter gene is selected from the group consisting of
an endogenous gene and the reporter substrate gene is introduced into the cells to be imaged; and
a gene introduced into the cells to be imaged, and the reporter substrate gene is an endogenous gene.

17. (canceled)

18. The method of claim 1, wherein the method is performed in vivo or in vitro.

19-20. (canceled)

21. The method of claim 2, wherein the enzyme is selected from the group consisting of CDase and herpes simplex virus type-1 thymidine kinase (HSV1-TK).

22-23. (canceled)

24. The method of claim 2, wherein the substrate is a nucleoside or synthetic nucleoside analog, optionally cytosine or 5-flurocytosine, pyrrolo-2′-deoxycytidine (pyrrolo-dC) or a synthetic thymidine analogue, optionally 5,6-dihydrothymidine or 5-methyl-5,6-dihydrothymidine.

25-26. (canceled)

27. The method of claim 2, wherein the enzyme is Drosophila melanogaster 2′-deoxynucleoside kinase (Dm-dNK).

28-31. (canceled)

32. The method of claim 16, wherein expression of the endogenous gene is induced.

33. (canceled)

34. A magnetic resonance imaging system comprising:
an imaging apparatus configured to perform a CEST MR technique; and
one or more reporter genes or reporter substrate genes.

35-44. (canceled)

45. A kit selected from the group consisting of
a kit for MRI imaging comprising one or more reporter genes or reporter substrate genes, and instructions for use; and
a kit for MRI imaging comprising one or more vectors expressing one or more reporter genes or reporter substrate genes, and instructions for use.

46. (canceled)

47. The method of claim 1, wherein the cells are from a subject suffering from a disease or disorder, optionally the disease or disorder is selected from the group consisting of: infectious diseases, neoplasms, endocrine, nutritional, and metabolic diseases, diseases of the blood and blood-forming organs, inflammatory diseases, immune diseases, including autoimmune diseases, diseases of the nervous system, diseases of the circulatory system, diseases of the respiratory system, diseases of the digestive system, diseases of the skin, diseases of the musculoskeletal system.

48. (canceled)

49. The method of claim 47, wherein the disease or disorder is selected from the group consisting of: cancer, diabetes and epilepsy.

50. The method of claim 1, wherein the MR imaging is performed in combination with positron emission tomography (PET).
